(19) United States
(12) Patent Application Publication
(10) Pub. No.: US 2024/0358947 A1
(43) Pub. Date: Oct. 31, 2024

(54) METHOD AND/OR APPARATUS FOR DETERMINING RESPIRATORY PARAMETERS

(71) Applicant: Fisher & Paykel Healthcare Limited a company incorporated in New Zealand with NZBN: 9429040555720, Auckland (NZ)

(72) Inventors: Thomas Heinrich BARNES, Surrey (GB); Matthew Jon PAYTON, Auckland (NZ); Rachael GLAVES, Auckland (NZ)

(21) Appl. No.: 18/685,631

(22) PCT Filed: Aug. 25, 2022
(86) PCT No.: PCT/IB2022/057947

§ 371 Date: Feb. 22, 2024

Related U.S. Application Data
(60) Provisional application No. 63/260,640, filed on Aug. 27, 2021.

(51) Int. Cl. A61M 16/00; A61M 16/10

(52) U.S. Cl. CPC A61M 16/024; A61M 16/1075; A61M 2016/0039; A61M 2205/3327

(57) ABSTRACT

A method of determining a respiratory parameter of a patient during inspiration when receiving respiratory support and their mouth is closed comprising: providing an apparatus gas flow with a flow rate and a gas proportion to a patient, measuring a gas proportion of a composite gas inflow to the patient, determining a composite gas inflow flow rate using one or more of: apparatus gas flow gas proportion, apparatus gas flow flow rate, composite gas inflow gas proportion, ambient gas proportion, and from the composite gas inflow flow rate, determining one or more respiratory parameters.

### FIELD OF THE INVENTION

The present invention relates to methods and/or apparatus for determining respiratory parameters.

### BACKGROUND TO THE INVENTION

When providing respiratory support, it is desirable to know various parameters, such as patient flow, tidal volume, minute ventilation, apnoea, respiratory rate, airway patency, peak flow rate. These cannot always be measured.

### SUMMARY OF INVENTION

It is an object of the invention to provide method and/or apparatus for determining one or more respiratory parameters, as described herein.

In one aspect the present invention may be said to comprise a method of determining a respiratory parameter of a patient during inspiration when receiving respiratory support and their mouth is closed comprising: providing an apparatus gas flow with a flow rate and a gas proportion to a patient, measuring a gas proportion of a composite gas inflow to the patient, determining a composite gas inflow flow rate using one or more of: apparatus gas flow gas proportion, apparatus gas flow flow rate, composite gas inflow gas proportion, ambient gas proportion, and from the composite gas inflow flow rate, determining one or more respiratory parameters.

Optionally the respiratory parameters are one or more of: tidal volume, minute ventilation, respiratory rate, apnoea, airway patency, and/or peak flow rate.

Optionally the apparatus gas flow flow rate is less than inspiratory demand of the patient for at least part of inspiration.

Optionally:
for some of inspiration, the apparatus gas flow is at a first flow rate that is at or above inspiratory demand, and
for some of inspiration, the apparatus gas flow is at a second or subsequent flow rate, each of the second or subsequent flow rate being below inspiratory demand.

Optionally the inspiratory demand is the inspiratory demand of the patient the method is performed on.

Optionally the apparatus gas flow flow rate is time-varying so that for some of inspiration the flow rate is at or above inspiratory demand and for some of inspiration the flow rate is below inspiratory demand.

Optionally the time-varying flow rate oscillates, and optionally oscillates at a frequency greater than breathing frequency.

Optionally the gas proportion is gas fraction and/or gas partial pressure.

Optionally the inspiratory demand is peak inspiratory demand.

Optionally the composite gas inflow comprises the apparatus gas flow and ambient (entrained) gas flow.

Optionally the gas is one or more of O2, CO2, N2 or a tracer gas.

Optionally the composite gas inflow gas proportion is measured with a sensor at the nose.

Optionally the method comprises determining the composite gas inflow flow rate using all of: apparatus gas flow gas proportion, apparatus gas flow flow rate, composite gas inflow gas proportion, ambient gas proportion.

Optionally the composite gas inflow flow rate Q_{TOT} of the patient is found using

$$\\ \begin{matrix}{Q}_{Tot} ( t ) = {Q}_{o} ( t ) [ 1 + \frac{Fo ( t ) - Fi {O}_{2} ( t )}{Fi {O}_{2} ( t ) - {F}_{entrained} ( t )} ] & ( 37 )\end{matrix}$$

where

Q_{TOT} the flow rate of the (total inhaled) patient gas inflow (composite gas inflow) 17 (apparatus gas flow 11 and entrained air gas flow 16) into the patient, and as defined by:

$$\\ \begin{matrix}{Q}_{Tot} ( t ) = {Q}_{entrained} ( t ) + {Q}_{o} ( t ) & ( 35 )\end{matrix}$$

and the state parameters are:

Qₒ is the flow rate of the apparatus gas flow

F_{O} is the volume fraction of the gas (e.g. O2, N2 or tracer gas) component in the apparatus gas flow coming from the respiratory apparatus

Fₘ is the volume fraction of the gas (e.g. O2, N2 or tracer gas) component in the composite gas inflow (patient inspiratory gas flow) to the patient

F_{entrained} is the volume fraction of the gas (e.g. O2, N2 or tracer gas) component in the entrained air gas flow.

Optionally wherein the tidal volume can be defined as follows

$$\\ \begin{matrix}{V}_{Tidal} ( t ) = \int {Q}_{Tot} ( t ) dt & ( 38 )\end{matrix}$$

In another aspect the present invention may be said to comprise a method of determining a respiratory parameter of a patient during expiration when receiving respiratory support and their mouth is closed comprising:
providing an apparatus gas flow with a flow rate and a gas proportion, to a patient, measuring a parameter of a gas present in a composite gas outflow from the patient, determining an exhaled gas flow rate using one or more of:
apparatus gas flow gas proportion, apparatus gas flow flow rate, composite gas outflow gas parameter,
exhaled gas flow parameter, wherein the exhaled gas flow parameter is determined using the measured parameter of the gas present in the composite gas outflow and a time-varying flow rate or gas proportion, and
from the exhaled gas flow rate, determining one or more respiratory parameters.

Optionally the respiratory parameters are one or more of: tidal volume, minute ventilation, respiratory rate, apnoea, airway patency, and/or peak flow rate.

Optionally the gas proportion is gas fraction and/or gas partial pressure.

Optionally the composite gas outflow comprises a leak gas flow and the exhaled gas flow.

Optionally the composite gas outflow parameter comprises a gas proportion measured with a sensor at the nose.

Optionally the gas is one or more of O2, CO2, N2 or a tracer gas.

Optionally the method comprises determining the exhaled gas flow flow rate using all of: apparatus gas flow gas proportion, apparatus gas flow flow rate, composite gas outflow gas proportion, exhaled gas flow parameter.

Optionally one of the flow rate and gas proportion are time-varying.

Optionally the apparatus gas flow flow rate or gas proportion oscillates.

Optionally the flow rate or gas proportion oscillates at a frequency greater than breathing frequency.

Optionally the exhaled gas flow parameter is an exhaled gas flow gas proportion.

Optionally the exhaled gas flow flow rate Q_{E} of the patient is found using:

$$\\ \begin{matrix}{Q}_{E} = {Q}_{O} ( t ) \frac{{F}_{O} ( t ) - {F}_{m} ( t )}{{F}_{m} ( t ) - {F}_{E} ( t )} & ( 41 )\end{matrix}$$

where
Q_{E} is the expiratory flow rate (exhaled gas-flow flow rate)
and the state parameters are
Qₒ is the flow rate of the apparatus gas flow
F_{O} is the volume fraction of the gas (e.g. O2, N2 or tracer gas) component in the apparatus gas flow 11 coming from the respiratory apparatus
Fₘ is the volume fraction of the gas (e.g. O2, CO2, N2 or tracer gas) component in the composite gas outflow 15 from the patient
F_{E} is volume fraction of the gas (e.g. CO2, O2, N2 or tracer gas) in the exhaled patient gas flow 13 (volume fraction of expired gas).

Optionally if using varying flow rate for apparatus gas flow and O2 fraction exhaled then

$$\\ \begin{matrix}{F}_{E} ( t ) \approx \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} & ( 4 )\end{matrix}$$

Optionally if using varying flow rate for apparatus gas flow and CO2 fraction exhaled then

$$\\ \begin{matrix}{F}_{E} ( t ) \sim {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{0} ( t ) {F}_{m} ( t )} & ( 16 )\end{matrix}$$

Optionally if using varying oxygen fraction for apparatus gas flow and O2 fraction exhaled

$$\\ \begin{matrix}{F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} & ( 17 )\end{matrix}$$

Optionally the tidal volume can be defined as follows

$${V}_{Tidal} ( t ) = \int {Q}_{E} ( t ) dt \qquad ( 42 )$$

In another aspect the present invention may be said to comprise a method of determining a respiratory parameter of a patient during expiration when receiving respiratory support comprising: providing an apparatus gas flow with a flow rate and a gas proportion, to a patient, measuring a parameter of the gas present in a composite gas outflow from the patient, determining a volume proportion of apparatus gas flow through the mouth and/or nose, determining an exhaled gas flow rate using one or more of: apparatus gas flow gas proportion, apparatus gas flow flow rate, composite gas outflow gas parameter, volume proportion of the apparatus gas flow, exhaled gas flow parameter, wherein the exhaled gas flow parameter is determined using the measured parameter of the gas present in the composite gas outflow and a time-varying flow rate or gas proportion of the apparatus gas flow, and from the exhaled gas flow rate, determining one or more respiratory parameters.

Optionally respiratory parameters are one or more of: tidal volume, minute ventilation, respiratory rate, apnoea, airway patency, and/or peak flow rate.

Optionally the gas proportion is gas fraction and/or gas partial pressure.

Optionally the composite gas outflow comprises a leak gas flow and the exhaled gas flow.

Optionally the composite gas outflow parameter is a gas proportion measured with a sensor at the mouth.

Optionally the gas is one or more of O2, CO2, N2 or a tracer gas.

Optionally the method comprises determining the exhaled gas flow flow rate using all of: apparatus gas flow gas proportion, apparatus gas flow flow rate, composite gas outflow gas proportion, exhaled gas flow parameter.

Optionally one of the flow rate and gas proportion are time-varying.

Optionally the apparatus gas flow flow rate or gas proportion oscillates.

Optionally the flow rate oscillates at a frequency greater than breathing frequency.

Optionally the exhaled gas flow parameter is an exhaled gas flow gas proportion.

Optionally determining a volume proportion of apparatus gas flow through the mouth and/or nose comprises determining a volume proportion of apparatus gas flow through the mouth.

Optionally the volume proportion of apparatus gas flow through the mouth is a constant k with a value between 0 and 1.

Optionally the exhaled gas flow flow rate Q_{E} of the patient is found using:

$${Q}_{E} ( t ) = k ( t ) {Q}_{o} ( t ) \frac{{F}_{o} ( t ) - {F}_{m} ( t )}{{F}_{m} ( t ) - {F}_{E} ( t )} \qquad ( 30 )$$

where

Q_{E} is the expiratory flow rate (patient exhaled gas-flow flow rate) and the state parameters are k is the proportion of the apparatus gas flow 11 that comes out through the mouth (and (1−k) is the fraction that goes through the nose). It is a value between 0 and 1, inclusive F_{O} is the volume fraction of the gas (e.g. O2, N2 or tracer gas) component in the apparatus gas flow 11 coming from the respiratory apparatus Fₘ is the volume fraction of the gas (e.g. O2, CO2, N2 or tracer gas) component in the composite gas outflow 15 from the patient F_{E} is volume fraction of the gas (e.g. CO2, O2, N2 or tracer gas) in the exhaled patient gas flow 13 (volume fraction of expired gas).

Optionally if using varying flow rate for apparatus gas flow and O2 fraction exhaled then

$${F}_{E} ( t ) \approx \frac{\begin{matrix}{Q}_{o} ( t ) {F}_{m} ( t + Δ t ) ( {F}_{o} ( t ) - {F}_{m} ( t ) ) - \\ {Q}_{o} ( t + Δ t ) {F}_{m} ( t ) ( {F}_{o} ( t + Δ t ) - {F}_{m} ( t + Δ t ) )\end{matrix}}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

Optionally if using varying flow rate for apparatus gas flow and CO2 fraction exhaled then

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

Optionally if using varying oxygen fraction for apparatus gas flow and O2 fraction exhaled

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

Optionally the tidal volume can be defined as follows

$${V}_{Tidal} ( t ) = \int {Q}_{E} ( t ) dt \qquad ( 42 )$$

In another aspect the present invention may be said to comprise a method of determining a respiratory parameter of a patient during expiration when receiving respiratory support comprising: providing an apparatus gas flow with a flow rate and a gas proportion, to a patient, measuring a parameter of the gas present in a composite gas outflow from the patient, determining an exhaled gas flow rate using one or more of: apparatus gas flow gas proportion, apparatus gas flow flow rate, composite gas outflow gas parameter, exhaled gas flow parameter, wherein the exhaled gas flow parameter is determined using the measured parameter of the gas present in the composite gas outflow and a time-varying flow rate or gas proportion of the apparatus gas flow, and from the exhaled gas flow rate, determining one or more respiratory parameters.

Optionally the method further comprises determining a volume proportion of apparatus gas flow through the mouth and/or nose, and wherein the exhaled gas flow parameter is determined using the measured parameter of the gas present in the composite gas outflow, a time-varying flow rate or gas proportion, and the proportion of apparatus gas flow through the mouth and/or nose.

In another aspect the present invention may be said to comprise a method of determining a respiratory parameter of a patient when receiving respiratory support comprising: determining in any order if a patient is inspiring or expiring and whether the mouth is open or closed, if the mouth is closed, during inspiration, determining a respiratory parameter according to one or more statements above, during expiration, determining a respiratory parameter according to according to one or more statements above, if the mouth is open, during expiration determining a respiratory parameter according to any one of claims 32 to 49, 50, 51

In another aspect the present invention may be said to comprise a respiratory support apparatus for providing respiratory support and determining a respiratory parameter comprising: a flow generator, one or more sensors or inputs for one or more sensors placed at a mouth and/or nose of a patient, a controller configured to carry out the method of any of claims 1 to 52.

Optionally the apparatus further comprises a humidifier.

Optionally the apparatus further has or connects to a non-sealing interface.

In another aspect the present invention can be said to comprise a method of determining a respiratory parameter of a patient during inspiration when receiving respiratory support and their mouth is closed comprising:
providing a non-therapeutic apparatus gas flow with a flow rate and a gas proportion to a patient,
measuring a gas proportion of a composite gas inflow to the patient,
determining a composite gas inflow flow rate using one or more of:
   apparatus gas flow gas proportion,
   apparatus gas flow flow rate,
   composite gas inflow gas proportion,
   ambient gas proportion, and
from the composite gas inflow flow rate, determining one or more respiratory parameters.

In another aspect the present invention can be said to comprise a method of determining a respiratory parameter of a patient during expiration when receiving respiratory support and their mouth is closed comprising:
providing a non-therapeutic apparatus gas flow with a flow rate and a gas proportion, to a patient,
measuring a parameter of the gas present in a composite gas outflow from the patient,
determining an exhaled gas flow rate using one or more of:
   apparatus gas flow gas fraction,
   apparatus gas flow flow rate,
   composite gas outflow parameter,
   exhaled gas flow parameter, wherein the exhaled gas flow parameter is determined using the measured parameter of the gas present in the composite gas outflow and a time-varying flow rate or gas proportion,
and
from the exhaled gas flow rate, determining one or more respiratory parameters.

In another aspect the present invention can be said to comprise a method of determining a respiratory parameter of a patient during expiration when receiving respiratory support comprising:
providing a non-therapeutic apparatus gas flow with a flow rate and a gas proportion, to a patient,
measuring a parameter of the gas present in a composite gas outflow from the patient,
determining a proportion of apparatus gas flow through the mouth and/or nose,
determining an exhaled gas flow rate using one or more of:
   apparatus gas flow oxygen fraction,
   apparatus gas flow flow rate,
   composite gas outflow parameter,
   volume proportion of gas flow,
   exhaled gas flow parameter, wherein the exhaled gas flow parameter is determined using the measured parameter of the gas present in the composite gas outflow and a time-varying flow rate or gas proportion,
and
from the exhaled gas flow rate, determining one or more respiratory parameters.

In another aspect the present invention can be said to comprise an apparatus for providing respiratory support and determining a respiratory parameter comprising:
a flow generator for providing an apparatus gas flow with a flow rate and a gas proportion to a patient,
one or more sensors or inputs for one or more sensors placed at a mouth and/or nose of a patient
a controller configured to when a patient is inspiring and the mouth is closed:
   measure a gas proportion of a composite gas inflow to the patient,
   determine a composite gas inflow flow rate using one or more of:
      apparatus gas flow gas proportion,
      apparatus gas flow flow rate,
      composite gas inflow gas proportion,
      ambient gas proportion, and
   from the composite gas inflow flow rate, determine one or more respiratory parameters.

An apparatus for providing respiratory support and determining a respiratory parameter comprising:
a flow generator for providing an apparatus gas flow with a flow rate and a gas proportion to a patient,
one or more sensors or inputs for one or more sensors placed at a mouth and/or nose of a patient
a controller configured to when a patient is expiring and the mouth is closed:
measure a parameter of the gas present in a composite gas outflow from the patient,
determine an exhaled gas flow rate using one or more of:
   apparatus gas flow gas fraction,
   apparatus gas flow flow rate,
   composite gas outflow parameter,
   exhaled gas flow parameter, wherein the exhaled gas flow parameter is determined using the measured parameter of the gas present in the composite gas outflow and a time-varying flow rate or gas proportion,
and
from the exhaled gas flow rate, determine one or more respiratory parameters.

In another aspect the present invention can be said to comprise an apparatus for providing respiratory support and determining a respiratory parameter comprising:
a flow generator for providing an apparatus gas flow with a flow rate and a gas proportion to a patient,
one or more sensors or inputs for one or more sensors placed at a mouth and/or nose of a patient
a controller configured to when a patient is expiring and the mouth is open:
measure a parameter of the gas present in a composite gas outflow from the patient,
determine a proportion of apparatus gas flow through the mouth and/or nose,
determine an exhaled gas flow rate using one or more of:
   apparatus gas flow oxygen fraction,
   apparatus gas flow flow rate,
   composite gas outflow parameter,
   volume proportion of gas flow,
exhaled gas flow parameter, wherein the exhaled gas flow parameter is determined using the measured parameter of the gas present in the composite gas outflow and a time-varying flow rate or gas proportion,
and
from the exhaled gas flow rate, determine one or more respiratory parameters.

In another aspect the present invention can be said to comprise an apparatus for providing respiratory support and determining a respiratory parameter comprising:
a flow generator for providing an apparatus gas flow with a flow rate and a gas proportion to a patient,
one or more sensors or inputs for one or more sensors placed at a mouth and/or nose of a patient

a controller configured to when a patient is expiring:
measure a parameter of the gas present in a composite gas outflow from the patient,
determine an exhaled gas flow rate using one or more of:
   apparatus gas flow gas fraction,
   apparatus gas flow flow rate,
   composite gas outflow parameter,
   exhaled gas flow parameter, wherein the exhaled gas flow

parameter is determined using the measured parameter of the gas present in the composite gas outflow and the time-varying flow rate or gas proportion,
and
from the exhaled gas flow rate, determine one or more respiratory parameters.

Optionally the controller is further configured to determine a proportion of apparatus gas flow through the mouth and/or nose, and wherein the exhaled gas flow parameter is determined using the measured parameter of the gas present in the composite gas outflow, a time-varying flow rate or gas proportion, and the proportion of apparatus gas flow through the mouth and/or nose.

Optionally, the apparatus has a humidifier Optionally the apparatus has or connects to a non-sealing interface.

In another aspect the present invention can be said to comprise a method of determining a respiratory parameter of a patient during inspiration when receiving respiratory support and their mouth is closed comprising: providing an apparatus gas flow with a flow rate and a gas proportion to a patient, measuring a gas proportion of a composite gas inflow to the patient, determining a composite gas inflow flow rate using one or more of: apparatus gas flow gas proportion, apparatus gas flow flow rate, composite gas inflow gas proportion, ambient gas proportion, wherein the apparatus flow flow rate is a time-varying flow rate that is below the patient inspiratory demand flow rate at least temporarily.

Optionally the time-varying flow rate is below the patient inspiratory demand when measuring the gas proportion of the composite gas inflow to the patient.

Optionally the time-varying flow rate is an oscillating flow rate.

Optionally the composite gas inflow flow rate indicates inspiratory demand flow rate.

In another aspect the present invention may be said to comprise an apparatus for providing respiratory support and determining a respiratory parameter comprising: a flow generator, one or more sensors or inputs for one or more sensors placed at a mouth and/or nose of a patient, a controller configured to carry out the method of any of the above paragraphs.

Optionally the apparatus further comprises a humidifier.

Optionally the apparatus has or connects to a non-sealing interface.

In another aspect the present invention may be said to comprise an apparatus for providing respiratory support and determining a respiratory parameter comprising: a flow generator, one or more sensors or inputs for one or more sensors placed at a mouth and/or nose of a patient, a controller configured to: control the flow generator to provide an apparatus gas flow with a time-varying flow rate and a gas proportion to a patient, receive target gas input from a target gas sensor, and determine an inspiratory demand flow rate based on: the target gas input, time-varying flow rate, gas proportion.

Optionally the apparatus further comprises a humidifier to humidify the apparatus gas flow.

Optionally the apparatus further comprises a non-sealing patient interface.

Optionally the target gas input relates to a target gas parameter.

Optionally:
the target gas is oxygen,
the target gas parameter is FiO2,
and/or
the target gas sensor is an O2 fraction sensor.

Optionally the apparatus flow flow rate is a time-varying flow rate that is below a patient inspiratory demand flow rate at least temporarily.

Optionally the controller receives an input indicative of a patient's respiratory phase, indicates an inspiration phase.

Optional the controller calculates tidal volume based on inspiratory flow rate.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed.

These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The phrase ‘computer-readable medium’ should be taken to include a single medium or multiple media. Examples of multiple media include a centralised or distributed database and/or associated caches. These multiple media store the one or more sets of computer executable instructions. The phrase ‘computer readable medium’ should also be taken to include any medium that is capable of storing, encoding or carrying a set of instructions for execution by a processor of a computing device and that cause the processor to perform any one or more of the methods described herein. The computer-readable medium is also capable of storing, encoding or carrying data structures used by or associated with these sets of instructions. The phrase ‘computer-readable medium’ includes solid-state memories, optical media and magnetic media.

In this specification where reference has been made to patent specifications, other external documents, or other sources of information, this is generally for the purpose of providing a context for discussing the features of the disclosure. Unless specifically stated otherwise, reference to such external documents is not to be construed as an admission that such documents, or such sources of information, in any jurisdiction, are prior art, or form part of the common general knowledge in the art.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth. The invention consists in the foregoing and also envisages constructions of which the following gives examples only.

### Definitions

Patient gas flow: This can be the gas flow into the patient (during inspiration—patient inspiratory gas flow) or the gas flow out of the patient (during expiration—patient expiratory gas flow).
In the case of inspiration where inspiratory demand is not met, it is a composite gas inflow 17 (also termed patient inspiratory gas flow or inspired gas flow or total gas inflow); that is—gas flow inspired by the patient) comprising the apparatus gas flow 11 and the ambient gas flow 16 (entrained air). In some cases, there may not be any entrainment of ambient air. The composite gas inflow has a gas fraction Fₘ. In the case of O2, the gas fraction is FiO2. If a sensor measures the O2 fraction during inhalation, it will be measuring (that is, Fₘ is) FiO2/the O2 fraction of the composite gas inflow.
In the case of inspiration where inspiratory demand is met, it may be apparatus gas flow 11, as no ambient gas flow 16 may be entrained.
In the case of inspiration where the apparatus gas flow 11 exceeds inspiratory demand, it might be a fraction of apparatus gas flow 11, as the excess apparatus gas flow (above inspiratory demand) might escape to ambient.
Note, because apparatus gas flow 11 can change between meeting, not meeting or exceeding inspiratory demand at various times, for simplicity, “composite gas inflow” can refer to any of the situations above, but depending on whether inspiratory demand is met, not met, or exceeded, the composite gas flow 17 might comprise ambient gas flow 16, not comprise ambient gas flow 16, or might not comprise all the apparatus gas flow 11.
In the case of expiration, it is the exhaled gas flow 13 (also termed patient expiratory gas flow; that is—gas flow expired by the patient) Apparatus gas flow 11: the gas flow from the respiratory apparatus.

Ambient gas flow 16: this is ambient air entrained into the patient's airways.

Composite gas inflow 17: as referred to in the patient gas flow definition,
In case where inspiratory demand is not met this is the combined inhaled apparatus gas flow 11 and the ambient gas flow 16 (entrained air).
In cases where inspiratory demand is met and there may be no entrainment, the composite gas inflow 17 is the apparatus gas flow 11.
In cases where gas apparatus flow exceeds inspiratory demand, the composite gas inflow 17 is a fraction of the apparatus gas flow 11.
Composite gas inflow 17 also can be referred to as “total patient gas inflow” or “total inhaled gas flow”

Exhaled gas flow 13: This is the gas exhaled by the patient, i.e. this is gas exiting from the patient's airways during expiration.

Leak gas flow 12: this comprises the excess gas flow from the apparatus gas flow 11 that is not inhaled and/or has not entered the lower airways of the patient by the patient and escapes to ambient via the mouth and/or nose.

Composite gas outflow 15 (also termed “total gas outflow”): This is the leak gas flow 12 combined with the exhaled gas flow 13.

All gas flows can have a parameter, for example a flow rate and/or a gas proportion. The proportion can be a gas fraction/concentration and/or a gas partial pressure. The parameter can be time-varying.

Reference to an instantaneous parameter, such as instantaneous flow rate or instantaneous gas fraction, refers to the value of that parameter in a gas flow at an instance in time.

State parameter: This is a parameter indicating the state of an apparatus, gas flow, patient or the like, such as, without limitation:
a) The state of the mouth, which might be a binary open/closed, or some parameter indicating the proportion of gas flow (e.g. volume proportion) from the apparatus gas flow exiting the mouth with respect to the nose. E.g. k
b) Flow rate of the gas flow from the respiratory apparatus. E.g. Qₒ
c) Gas fraction (which includes any equivalent gas proportion) of the gas flow from the respiratory apparatus. E.g. O2 fraction but could be any other gas proportion where suitable, e.g. N2 or tracer gas F_{O}, but could be any other gas proportion where suitable, e.g. N2 or tracer gas
d) Gas fraction of gas flow into or out of the patient (depending if measured in inhalation or exhalation). E.g. O2 fraction Fₘ but could be any other gas proportion where suitable, e.g. CO2, N2 or tracer gas
e) Gas fraction of exhaled gas flow by the patient. F_{E} could be any gas proportion where suitable, e.g. O2, CO2, N2 or tracer gas
f) Gas fraction and flow rate of entrained (ambient) gas. Qₑₙₜ, Fₑₙₜ (note, can also be referred to as F_{entrained} which will be used interchangeably herein). But could be any gas proportion where suitable, e.g. O2, CO2, N2 or tracer gas
g) Flow rate of patient gas flow (which can be a respiratory parameter as well as a state parameter) being the:
   a. flow rate Q_{E} of exhaled gas flow 13 (that is—gas flow expired by the patient), and/or
   b. flow rate Qₜₒₜ of composite gas inflow 17 (that is—composite gas flow inspired by the patient). Note, Q_{TOT} also indicates the inspiratory demand flow rate of the patient

Above, the gas fraction could be the O2, CO2 or other gas fraction. Gas partial pressure could be used instead of gas fraction, and that will be understood as interchangeable by skilled person throughout this specification. That is, any reference to gas fraction could be a reference to gas partial pressure.

Respiratory parameter: This is a parameter indicating the state of respiration, such as, without limitation
Tidal volume: The amount of air that moves in or out of the lungs with each respiratory cycle, usually measured in ml.
Minute ventilation: The amount of air breathed per minute, usually measured in litres.
Respiratory rate: The rate at which breathing occurs, usually measured in breaths per minute.
Apnoea: Cessation of breathing, which could be temporary.
Airway patency.
Peak flow rate.

State parameters are used to determine respiratory parameters. There can be cross over between state parameters and respiratory parameters.

In this specification, reference to “exhale” can be used interchangeably with “expire”

In this specification, reference to “proportion” in the context of gas refers to any relative measure of a constituent gas component in a total gas comprising two or more constituent gas components. For example, proportion could cover:
volume fraction,
fraction,
volume concentration,
concentration,
molarity,
partial pressure

The proportion measured may be the parameter that is measured by the sensor being used, be it concentration, fraction, partial pressure or otherwise. The proportion determined may be the parameter desired by a user and/or processed by a component of a respiratory system or associated with a respiratory system.

In this specification, reference to “concentration” can also be termed “fraction” and can be indicated as percentage by volume of the gas of interest versus the volume of constituent gases overall in the gas flow in question, be it exhaled gas flow, apparatus flow or any other flow. However, the parameter could be a different measure and the gas could be different—these are just examples.

The gas relating to the gas parameter being determined could be, without limitation, oxygen (O2), carbon dioxide (CO2), nitrogen (N), helium (He) or Sevoflurane. Where reference is made to a particular gas herein, it will be appreciated that it is by way of example only and the description can apply to any gas—not just that referenced.

In this specification, “high flow” means, without limitation, any gas flow with a flow rate that is higher than usual/normal, such as higher than the normal inspiration flow rate of a healthy patient. It can be provided by a non-sealing respiratory system with uncontrolled and often substantial leak happening at the entrance of the patient's airways due to a non-sealing patient interface, for example non-sealing prongs. It can be also provided with humidification to improve patient comfort, compliance and safety. Alternatively or additionally, it can be higher than some other threshold flow rate that is relevant to the context—for example, where providing a gas flow to a patient at a flow rate to meet inspiratory demand (e.g. instantaneous inspiratory demand or peak inspiratory demand—this could be the inspiratory demand of the patient that is receiving the respiratory support, or a representative inspiratory demand, such as representative of patients based on e.g. empirical data), that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. Those skilled in the art know from context what constitutes “high flow”. It is a magnitude of flow rate that is over and above a flow rate that might otherwise be provided.

But, without limitation, some indicative values of high flow can be as follows.
In some configurations, delivery of gases to a patient at a flow rate of greater than or equal to about 5 or 10 litres per minute (5 or 10 LPM or L/min).
In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source, may comprise, but is not limited to, flows of at least about 5, 10, 20, 30, 40, 50, 60, 70, 80, 90,100,110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a procedure/therapy/support. Gases delivered may comprise a percentage of oxygen. In some configurations, the percentage of oxygen in the gases delivered may be about 15% to about 100%, about 20% to about 100%, or about 21% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The therapeutic flow rate can be set to 0.4-0.8 L/min/kg with a minimum of about 0.5 L/min and a maximum of about 70 L/min. For patients under 2 kg maximum flow is set to 8 L/min.

The oscillating flow is set to 0.05-2 L/min/kg with a preferred range of 0.1-1 L/min/kg and another preferred range of 0.2-0.8 L/min/kg.

The therapeutic flow rate can be time-varying (e.g. oscillating)—that is, the therapeutic flow can have a time-varying (e.g. oscillating) flow rate component. This time-varying flow rate can help with respiratory support.

Note, the embodiments herein also have signature flow rates that are time-varying (e.g. oscillate) and might be in addition to the therapeutic flow rates. Therefore, where therapeutic time-varying flow rates are used, the gas flow rate from the apparatus will have a therapeutic time-varying gas flow component(s) (portion) and a signature time-varying flow rate component(s) (portion). Therapeutic time-varying flow rates have a different purpose to signature time-varying flow rates, and might be different frequencies and/or amplitudes (although, they could overlap or be the same). Signature flow rates could be lower, the same or higher than therapeutic flow rates. Signature flow rate frequencies could be lower, the same or higher than therapeutic flow rate frequencies (when time-varying). In some embodiments, the signature flow rate has a higher frequency than the therapeutic flow rate. Therapeutic (constant or time-varying) flow rates are to provide respiratory support, airway clearance, oxygenation or the like whereas signature time-varying flow rates are to assist with determining a gas parameter. Signature time-varying flow rates will be described in more detail later. Throughout the specification, unless otherwise stated, the focus will be on the signature time-varying flow rates, but that does not exclude that there might also be therapeutic time-varying flow rates for therapeutic reasons.

As an example, the signature flow rates can step between a first flow rate and second flow rate, one or both of which can fall in the range of about 0 LPM to 70 LPM. The maximum signature flow rate could be the therapeutic flow rate. The signature flow rate could be combined with the therapeutic flow rate (e.g. added) or it could form part or all of the therapeutic flow rate—that is, the therapeutic flow rate itself could be the signature flow rate. In some embodiments, the signature flow rate can be related to the therapeutic flow rate as a percentage. For example, the signature time-varying flow rate (adults) is in the range of:
about 0% to about 200% of the therapeutic flow rate,
about 0% to 100% of the therapeutic flow rate,
about 100% to 200% of the therapeutic flow rate, or
about 50% to 150% of the therapeutic flow rate, and/or is in the range of
about 0-140 LPM,
about 0-70 LPM,
about 70-140 LPM,
about 40-100 LPM, or
about 20-60 LPM

These are not limiting flow rates, and it should also be noted that the signature flow rates could be negative, but when combined with the therapeutic flow rates create a total flow rate that is positive.

High flow has been found effective in meeting or exceeding the patient's normal real inspiratory flow, to increase oxygenation of the patient and/or reduce the work of breathing. Additionally, high flow may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

### BRIEF DESCRIPTION OF DRAWINGS

Embodiments will be described with reference to the following drawings, of which:

FIG. 1A shows provision of apparatus gas flow, and the patient and apparatus gas flows, flow rates and gas fractions that occur

FIG. 1B shows a diagram of a general embodiment of a respiratory support apparatus.

FIG. 2 shows a flow diagram of an overview of a method for determining respiratory parameters FIGS. 3 to 5 show flow diagrams of different examples of methods for determining respiratory parameters.

FIG. 6 shows a flow diagram of a combined method of determining respiratory parameters.

FIG. 7 shows a flow diagram of a particular example of a combined method of determining respiratory parameters.

FIG. 8 shows one embodiment of an apparatus for delivering respiratory support and implementing one or more of the methods for determining a respiratory parameter.

### DETAILED DESCRIPTION

### 1. Overview

The present embodiments relate to methods and/or apparatus for determining one or more respiratory parameters of a patient receiving respiratory support, comprising, but not limited to:
Flow rate of patient gas flow
Tidal volume
Minute ventilation—tidal volume can be determined for each breath within a minute time period from which the minute ventilation is calculated
Respiratory rate—e.g. peak to peak detection based on the calculated tidal volume/flows, e.g. rolling count of breaths per minute. Respiratory rate could also be measured with a zero-crossing method (instead of peaks)
Apnoea—when the patient is not breathing there may be no changes to the measured gas concentrations due to contribution from patient or entrainment. Thus, when there is apnoea, sensors may only measure delivered gas flows at all times.
Airway patency—Determination of airway patency.
   One technique is to monitor flow and/or oxygen oscillations at the mouth from the high flow system. If they can be seen then we know:
      The high flow is in place
      That the nasal passage is patent.
   if the oscillations do not match what is being applied at the nose (that is they are being modulated) then we can conclude that:
      the patient is breathing and
      That the lower airway is patent.
   This may be combined with measurements of FeCO2 to confirm:
      The patient is breathing and
      That the lower airway is patent.
Peak flow rate.

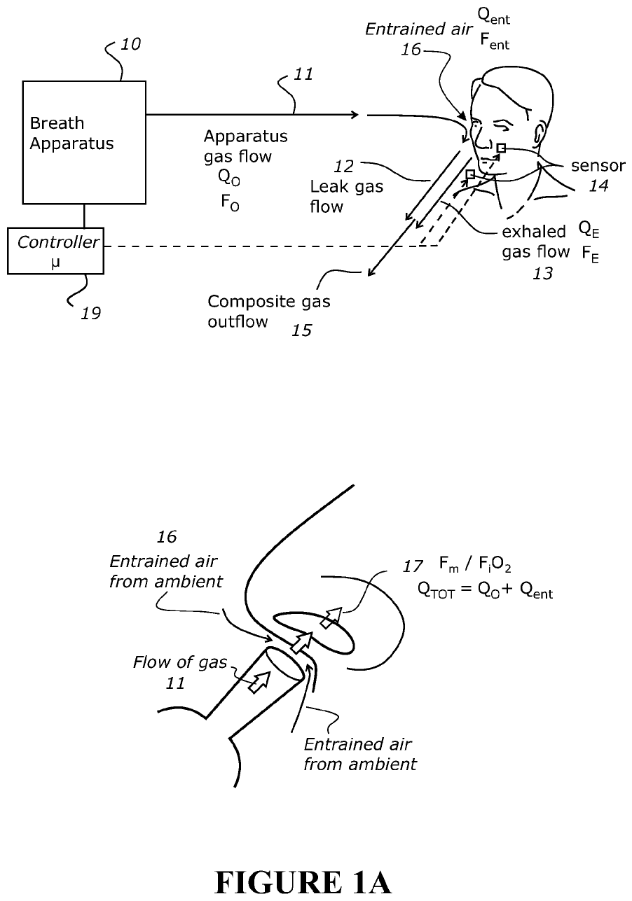

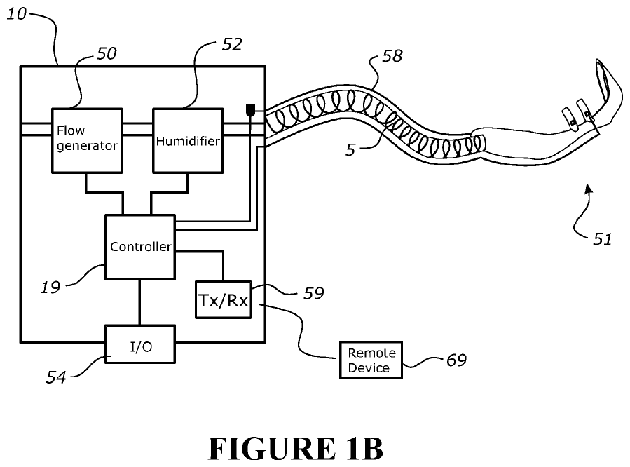

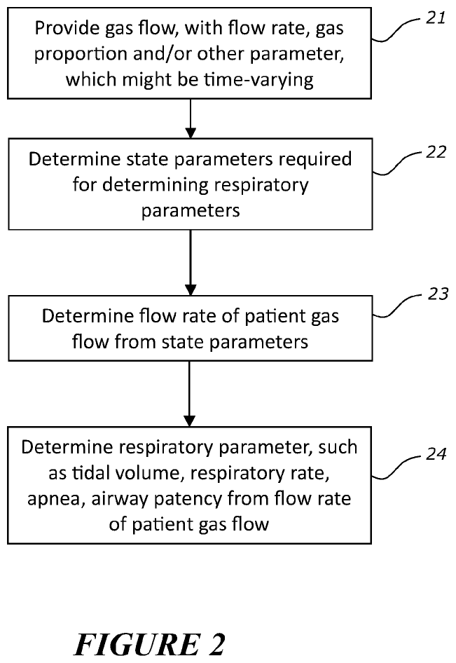

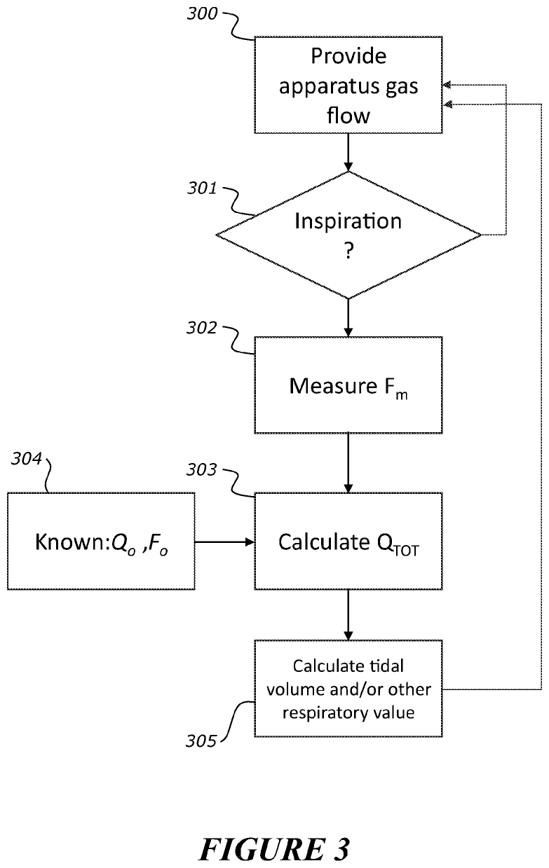

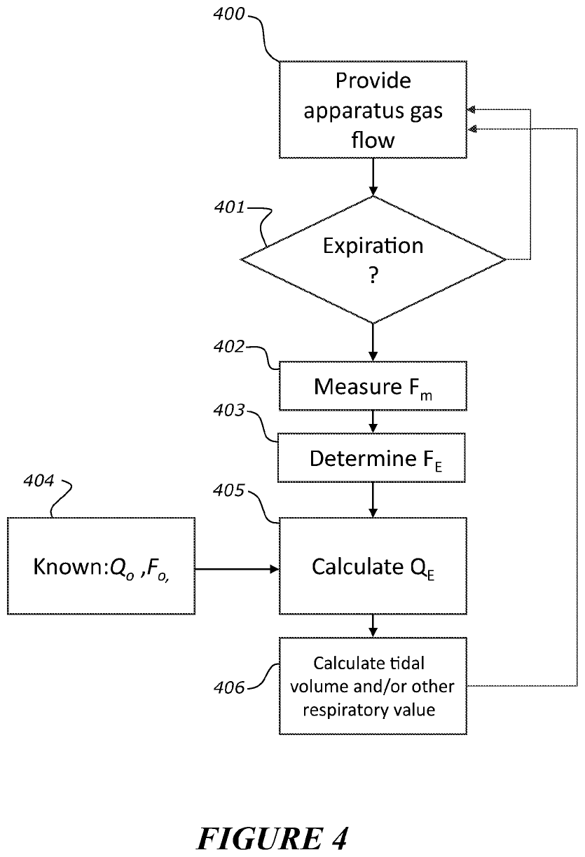

Referring to FIGS. 1A, 1B and 2, in the context of the patient receiving respiratory support from a respiratory apparatus in the form of a gas flow with a flow rate and oxygen fraction (one or both of which might be constant (i.e. set flow rate and/or O2 fraction) or varying), the method (FIG. 2) and/or apparatus (FIGS. 1A, 1B) determine one or more of the respiratory parameters using some combination of the following information (state parameters)
a) The state of the mouth, which might be a binary open/closed, or some parameter indicating the proportion of gas flow (e.g. volume proportion) from the apparatus gas flow exiting the mouth with respect to the nose. E.g. k
b) Flow rate of the gas flow from the respiratory apparatus. E.g. Qₒ
c) Gas fraction (which includes any equivalent gas proportion) of the gas flow from the respiratory apparatus. E.g. O2 fraction but could be any other gas proportion where suitable, e.g. N2 or tracer gas F_{O}, but could be any other gas proportion where suitable, e.g. N2 or tracer gas
d) Gas fraction of gas flow into or out of the patient (depending if measured in inhalation or exhalation). E.g. O2 fraction Fₘ but could be any other gas proportion where suitable, e.g. CO2, N2 or tracer gas
e) Gas fraction of exhaled gas flow by the patient. F_{E} could be any gas proportion where suitable, e.g. O2, CO2, N2 or tracer gas
f) Gas fraction and flow rate of entrained (ambient) gas. Qₑₙₜ, Fₑₙₜ (note, can also be referred to as F_{entrained} which will be used interchangeably herein). But could be any gas proportion where suitable, e.g. O2, CO2, N2 or tracer gas
g) Flow rate of patient gas flow (which can be a respiratory parameter as well as a state parameter) being the:
   a. flow rate Q_{E} of exhaled gas flow 13 (that is—gas flow expired by the patient), and/or
   b. flow rate Qₜₒₜ of composite gas inflow 17 (that is—composite gas flow inspired by the patient). Note, Q_{TOT} also indicates the inspiratory demand flow rate of the patient

Above, the gas fraction could be the O2, CO2, N2 or other gas fraction (or more generally gas proportion where proportion is understood to be equivalent alternatives to fraction). For example, partial pressure could be used instead of gas fraction, and that will be understood as interchangeable by a skilled person throughout this specification.

The above might be known, measured, calculated from one or more of the other state parameters or otherwise determined.

In general terms (see FIG. 2), in one example, a state parameter g) (the flow rate of the patient gas flow—either the flow rate of gas expired by the patient, or the flow rate of gas inspired by the patient) is calculated from a combination of one or more state parameters a)-f). The flow rate of patient gas flow (either Q_{TOT} or Q_{E}) is then integrated over one inspiration or expiration cycle to obtain tidal volume (respiratory parameter). The flow rate of the patient gas flow could also be an outcome in its own right (that is, a respiratory parameter) as well as being a state parameter. In which case, further respiratory parameters might not be derived from Q_{E}/Q_{TOT} but rather, they become the required respiratory parameters.

More generally, the method is set out in FIG. 2 where gas flow from a respiratory apparatus is provided to a patient with a flow rate and an oxygen fraction (or other gas proportion), one or both of which might be time-varying, step 21. One or more state parameters are known, measured, or calculated, or otherwise determined step 22. The flow rate of the patient gas flow of the patient is determined from the state parameters, step 23, and then one of the respiratory parameters are calculated, step 24.

An overview of implementing the method on an apparatus will now be described with reference to FIGS. 1A, 1B. By way of example, a high flow respiratory apparatus 10 is described with reference to e.g. FIGS. 1A and 1B. It will be noted that a high flow respiratory apparatus is used by way of example only, and the embodiment herein can work with low flow or any other type of suitable respiratory apparatus. References herein to a high flow respiratory apparatus and related parameters are exemplary only and should not be considered limiting as to what respiratory support the embodiments could be used with. In general terms, the apparatus comprises a main housing 10 that contains a flow generator 50 which may be in the form of a motor/impeller arrangement (or alternatively other flow/modulator sources and/or valves), an optional humidifier 52, a controller 19, and a user I/O interface 54 (comprising, for example, a display and input device(s) such as button(s), a touch screen, or the like). The apparatus can include one or more communication modules 59 to enable data communication or connection with one or more external devices or servers over a data or communication link or data network, whether wired, wireless or a combination thereof. In one configuration for example, the apparatus 10 can include a wireless data transmitter and/or receiver, or a transceiver 59 to enable the controller 19 to receive data signals in a wireless manner from the operation sensors and/or to control the various components of the system 10. The controller 19 is configured or programmed to control the components of the apparatus, including: operating the flow generator to create a flow of gas (gas flow) for delivery to a patient, operating the humidifier (if present) to humidify and/or heat the generated gas flow, receive user input from the user interface for reconfiguration and/or user-defined operation of the apparatus, output information (for example on the display) to the user and transmit and/or receive information via the communication module 59 to a remote device 69. The user could be a patient, healthcare professional, or anyone else interested in using the apparatus. A patient breathing conduit 58 is coupled to a gas flow output in the housing of the respiratory apparatus, and is coupled to a (e.g. non-sealing) patient interface 51 such as a (e.g. non-sealing) nasal cannula with a manifold and nasal prongs. The patient breathing conduit can have a heater wire 5 to heat gas flow passing through to the patient.

High flow may be used as a means to promote gas exchange and/or respiratory support through the delivery of oxygen and/or other gases, and through the removal of CO2 from the patient's airways. High flow may be particularly useful prior to, during, or after a medical procedure.

Further advantages of high gas flow can comprise that the high gas flow increases pressure in the airways of the patient, thereby providing pressure support that opens airways, the trachea, lungs/alveolar and bronchioles. The opening of these structures enhances oxygenation, and to some extent assists in removal of CO2.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

Referring to FIG. 1A, the present embodiments can be used in any suitable situation where a patient is provided a gas flow from a respiratory apparatus 10 for providing therapy (respiratory support) (such as, a high gas flow for, but not limited to, high flow therapy). The apparatus 10 provides an apparatus gas flow 11. This apparatus gas flow 11 has a flow rate Qₒ. The flow rate might be a constant flow rate (that is not varying over time), or it might be time-varying, depending on the requirements of therapy. The apparatus gas flow may also have an O2 fraction Fₒ(as an example, but could be another gas fraction, and noting more generally it could be a gas proportion, but fraction is used here for exemplary purposes). The O2 fraction might be constant (that is not varying over time), or it might be time-varying, depending on the requirements. In these situations, the patient will breathe at least some of the apparatus gas flow 11, during inspiration. Also, the patient might entrain air, as an entrained gas flow 16. The entrained gas flow has a flow rate and O2 fraction—Qₑₙₜ, Fₑₙₜ. The total patient gas inflow (also termed “composite gas inflow”, or “total inhaled gas flow”) 17 is the combination of the entrained gas flow and the apparatus gas flow 11 (where inspiratory demand is not met, but as explained earlier it might just be apparatus gas flow 11 if inspiratory demand met or even just a fraction of apparatus gas flow 11 if inspiratory demand exceeded. From this point on, that distinction will be understood by skilled person, even if not explicitly mentioned). In cases where there is no entrainment, the composite gas inflow 17 is the apparatus gas flow 11. The total flow rate is Q_{TOT}=Qₒ+Qₑₙₜ, which also indicates the inspiratory demand flow rate of the patient There is also a combined O2 fraction Fₘ (also referred to as FiO2 during inspiration).

The patient will exhale gas flow 13, with flow rate Q_{E} and O2 fraction F_{E}. This might come from the mouth and/or nose. The exhaled gas flow 13 will have constituent gas components, such as CO2, O2, Nitrogen, Helium and the like. The exhaled gas flow 13 may also comprise anaesthetic agents, such as sevoflurane.

There is also “Leak gas flow” 12, which comprises the excess gas flow from the apparatus gas flow 11 that is not inhaled and/or has not entered the lower airways of the patient and escapes to ambient via the mouth and/or nose.

“Composite gas outflow” (also termed “total gas outflow”) 15 is the leak gas flow 12 combined with the exhaled gas flow 13.

Exhaled gas flow 13, leak gas flow 12 and the resulting composite gas outflow 15 can come out of the mouth, the nose, or the mouth and nose. There are several scenarios: 1) A patient's mouth is open and the exhaled gas flow, the leak gas flow and therefore resulting composite gas outflow are predominantly (a term which can comprise/encompass entirely) coming out of the patient's mouth. 2) A patient's mouth is open and the exhaled gas flow, the leak gas flow and therefore resulting composite gas outflow are coming out of both the patient's mouth and nose. 3) A patient's mouth is closed and the exhaled gas flow, the leak gas flow and therefore resulting composite gas outflow are coming out of the patient's nose.

When measuring the composite gas outflow 15, this can be done with a suitable sensor. The sensor can be placed anywhere suitable, but its sensing portion (sensor input) can be, either placed to measure flow out the mouth, flow out the nose, or flow out the nose and mouth. Various implementations of sensors are possible, and while a sensor can be placed anywhere suitable, when referencing position relative to mouth and/or nose, that is an indication of where the sensing portion (sensing input) is positioned. For example, in a sensor with a sampling conduit, the sampling conduit (sensing portion) is near mouth and/or nose, even if the sensor or parts of sensor are elsewhere. Other implementations of sensor will be know to those skilled in the art too. Where the sensor is measuring flow just out the mouth, or just out the nose, the sensor might not be measuring the entire composite gas outflow, as some might also be exiting the other orifice (e.g. the other of the nose or mouth depending on which the sensor is not measuring). In this case, the sensor measurement is still suitable and/or obtaining enough of a measure of composite gas outflow for a determination of the gas parameter of the exhaled gas flow.

The present embodiments utilise methods and/or apparatus of PCT/IB2021/052062, U.S. application 62/989,081 (from which PCT/IB2021/052062 claims priority), PCT/IB2021/051587 and US application U.S. 62/982,298 (from which, PCT/IB2021/051587 claims priority), all of which are incorporated herein by reference in their entirety.

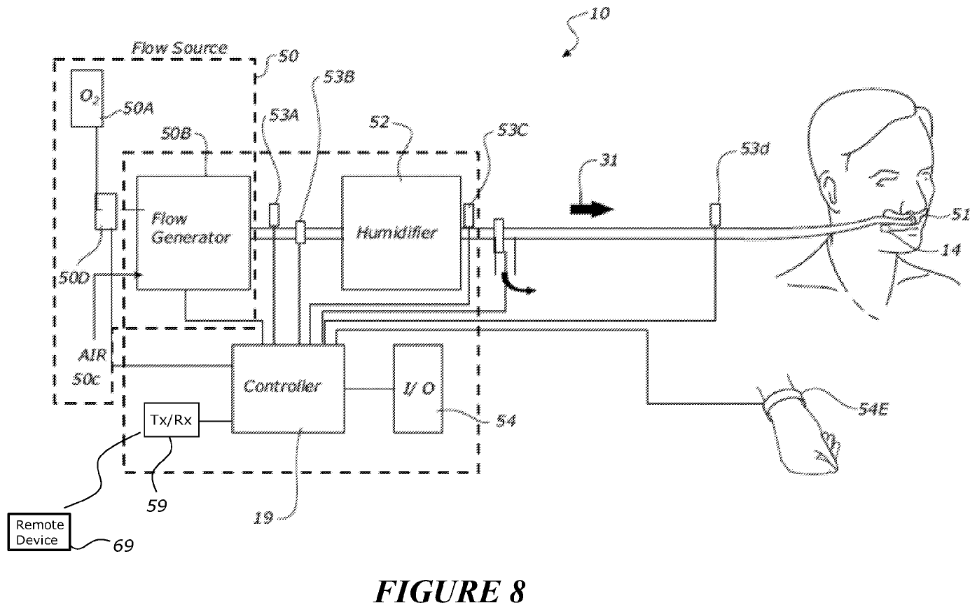

Three exemplary but non-limiting embodiments of a method will now be described in further detail with reference to FIGS. 3 to 6. Such embodiments can be carried out, for example, in the apparatus of FIG. 1B or 8.

Referring to FIG. 3, in one example, the tidal volume and/or other respiratory parameters can be determined from a patient with a closed mouth during an inspiratory portion of patient gas-flow, as can be seen in steps 300 to 305. Fₘ is measured/determined, Qₒ, F_{O} are known, Q_{TOT} can be calculated, and from that tidal volume and/or any respiratory value can be calculated or otherwise determined.

Referring to FIG. 4, in one example, the tidal volume and/or other respiratory parameters can be determined from a patient with a closed mouth during an expiratory portion of patient gas-flow, as can be seen in steps 400 to 406. F_{E}, Fₘ are measured/determined, Qₒ, F_{O} are known, Q_{E} can be calculated, and from that tidal volume and/or any respiratory value can be calculated or otherwise determined.

Referring to FIG. 5, in one example, the tidal volume and/or other respiratory parameters can be determined from a patient with an open mouth during an expiratory portion of patient gas-flow, as can be seen in steps 400 to 406, 500. K, F_{E}, Fₘ are measured,/determined Qₒ, F_{O} are known, Q_{E} can be calculated, and from that tidal volume and/or any respiratory value can be calculated or otherwise determined.

In any of the above, it is possible that patient inspiratory gas flow flow rate (Q_{TOT}) or a patient expiratory gas flow flow rate (Q_{E}) become the respiratory parameter output in its own right, and no further calculation is made for tidal volume or other respiratory parameter. That is, steps 305, 405, 505 could be omitted.

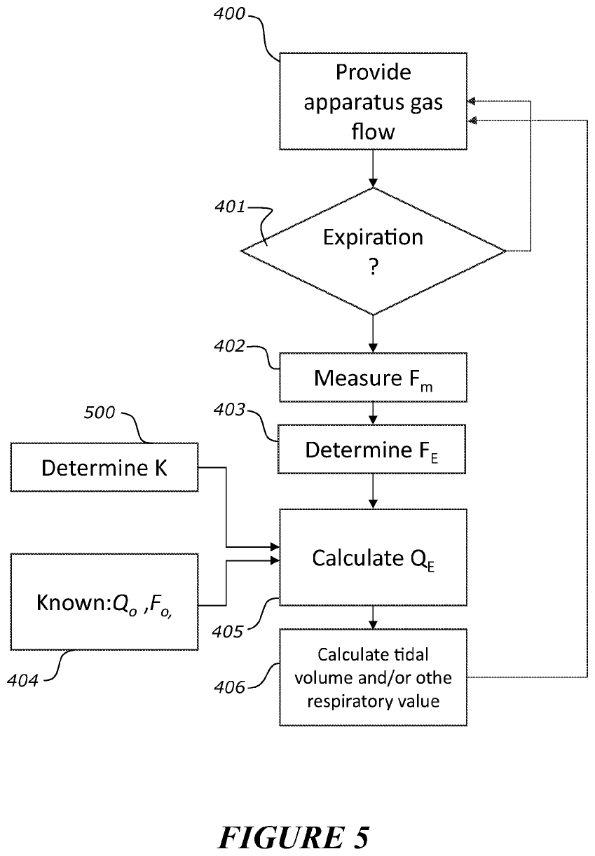

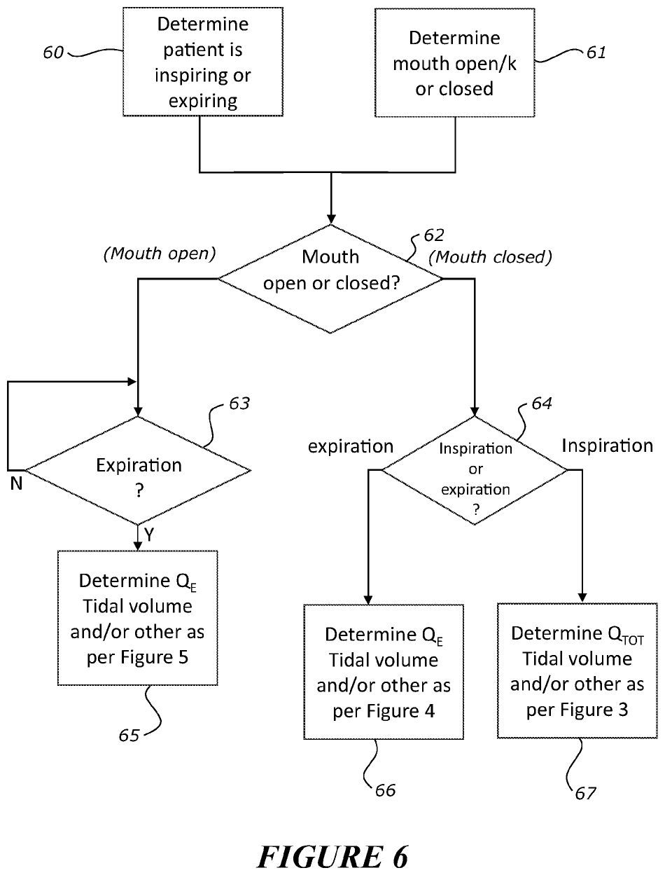

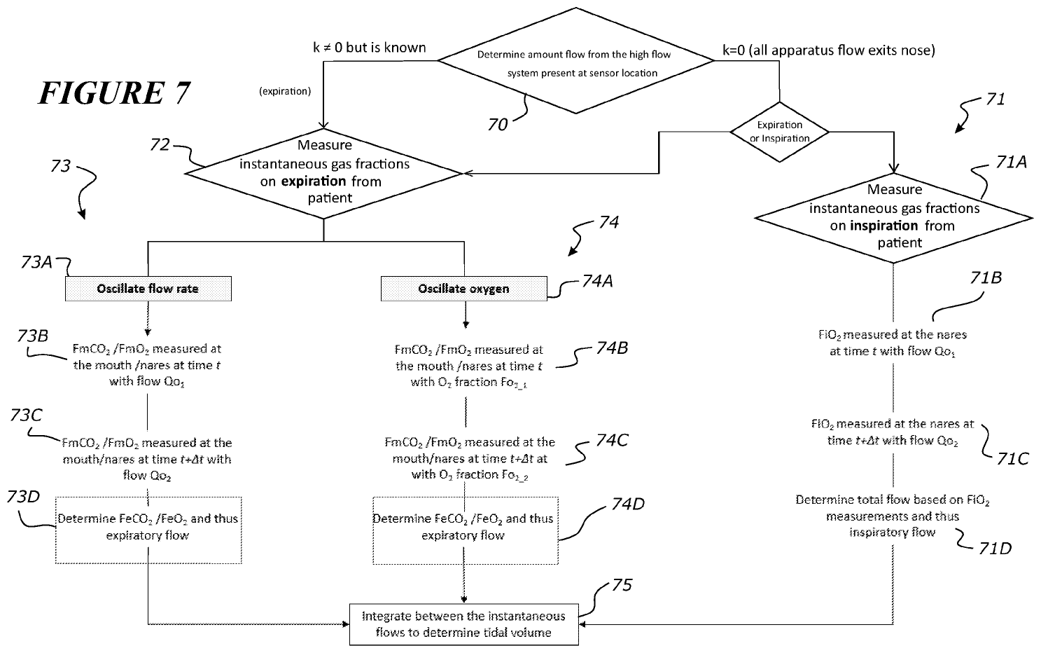

FIG. 6 shows how the three embodiments might be used jointly in a system. In each case, the method involves determining a patient inspiratory gas flow flow rate (Q_{TOT}) or a patient expiratory gas flow flow rate (Q_{E}) using some combination of the state parameters as per the FIGS. 3 to 5 and as per the description above and in more detail below. It can also involve then using the patient inspiratory gas flow flow rate or the patient expiratory gas flow flow rate to determine one or more additional respiratory parameters. FIG. 7 shows an example of the methods used jointly, being one example of the more general FIG. 6 method (and of which there are other possible examples).

The embodiments will now be described in more detail with reference to FIGS. 3 to 7.

2. Determining Composite Gas Inflow Flow Rate and/or Other Respiratory Parameters During the Inspiratory Cycle when the Patient Mouth is Closed.

Referring to FIG. 3, in one embodiment, a patient inspiratory gas flow flow rate is determined from a combination of state parameters, and then from that optionally one or more of the respiratory parameters are found. The method of this embodiment is carried out when the patient's mouth is closed, during patient inspiration. The patient's mouth closed can be determined in any suitable way, by a person and/or apparatus. For example, a person might look at a patient and see their mouth is closed, and provide input to an apparatus of the mouth state being closed.

The mouth being closed is a special case of the constant k=0. k is a value between 0 and 1 and is the proportion of delivered gases (apparatus gas flow 11) going through the mouth (e.g. in one example, when k=0, no apparatus gas flow 11 comes out of (exits) the mouth). If the mouth is closed as in this case, k=0, as no apparatus gas flow 11 goes through the mouth. In this case k is constant as the mouth is closed all the time. In the more general case, k might vary over time as the mouth “openness” varies over time. In this case, the embodiment later relating to the mouth and nose being open is relevant, and k can become depending on t, that is k(t). In that event, k it a time variable constant, that is a constant in a mathematical equation sense, but which can vary so is not a constant value over time. It should be noted that even in the open mouth situation, k might not be dependent on time, so both time dependent and non-time dependent k is possible. In the case where the mouth is closed (k=0), the equations are based on Fₘ being measured through a sensor 14 sensing at the nose.

The constant k will be described further in relation to embodiments where the mouth is not closed.

The method is carried out on an apparatus, such as described below. The full derivation of the equations used by the method is set out in the derivations section below.

In summary, the composite gas inflow (patient inspiration) 17 flow rate Q_{TOT} of the patient is found using

$${Q}_{Tot} ( t ) = {Q}_{o} ( t ) [ 1 + \frac{Fo ( t ) - {FiO}_{2} ( t )}{{FiO}_{2} ( t ) - {F}_{entrained} ( t )} ] \qquad ( 37 )$$

where

Q_{TOT} the flow rate of the (total inhaled) patient gas inflow (composite gas inflow) 17 (apparatus gas flow 11 and entrained air gas flow 16) into the patient, and as defined by:

$${Q}_{Tot} ( t ) = {Q}_{entrained} ( t ) + {Q}_{o} ( t ) \qquad ( 35 )$$

and the state parameters are:
Qₒ is the flow rate of the apparatus gas flow 11
F_{O} is the volume fraction of the gas (e.g. O2, N2 or tracer gas) component in the apparatus gas flow 11 coming from the respiratory apparatus.
Fₘ is the volume fraction of the gas (e.g. O2, N2 or tracer gas) component in the composite gas inflow (patient inspiratory gas flow) to the patient. As this is during inspiration, Fₘ is FiO2.
F_{entrained} is the volume fraction of the gas (e.g. O2, N2 or tracer gas) component in the entrained air gas flow 16.

Note reference to O2 herein with reference to the above parameters could also be interchanged with CO2, N2 or other tracer gas instead. Also more generally, any gas proportion could be used.

Once Q_{TOT} is calculated it can be used to find the tidal volume (a respiratory parameter) by integrating Q_{TOT} over the inspiration cycle. The tidal volume can be defined as follows.

$${V}_{Tidal} ( t ) = \int {Q}_{Tot} ( t ) dt \qquad ( 38 )$$

In addition other respiratory parameters can be found as follows.
Minute ventilation
Respiratory rate
Apnoea
Airway patency
Peak flow rate

Referring to the flowchart in FIG. 3, the method will now be described in further detail.

Qₒ, F_{O} are known operating parameters of the apparatus/apparatus gas flow 11, step 304. Likewise, F_{entrained} is the gas fraction of oxygen in ambient air, and known —approximately 21%. This means, to determine Q_{TOT} from equation 37, FiO2 needs to be determined.

An apparatus gas flow 11 is generated and provided by the respiratory apparatus 10 towards the patient via a patient interface 51, step 300. During the inspiration cycle of the patient, step 301, Fₘ (in this case FiO2 as it is during inspiration) is measured, step 302, by a suitable sensor 14, for example an oxygen fraction sensor 14. The sensor 14 may be arranged approximate the nares of the patient and/or in the nares of the patient. In the case of the mouth being open, a mouth sensor is used, whereas in the case of the mouth being closed (in this example), a nose sensor is used—this is explained further later with respect to the embodiments. For example, the oxygen fraction sensor 14 could be on the cannula 51 providing the apparatus gas flow 11.

The oxygen sensor measures the oxygen fraction Fₘ of the patient gas flow 17 entering (being inspired) the patient's airways. The measurement could be taken at time intervals (e.g. continuously or at least periodically/discretely) over time during an inspiration cycle. For example, the measurement can be taken at Fₘ(t), Fₘ(t+Δt) etc. (that is at time t and at some time later) This provides multiple measurements, step 302, along with Qₒ,F_{O}, F_{entrained} which are known, step 304, which can result in a corresponding Q_{TOT} (t), Q_{TOT}(t+Δt) etc. values calculated at those time intervals using the equation 37, step 303. This provides a time-dependent series of Q_{TOT} (t) which can be integrated to obtain the tidal volume, using equation 38, step 305. Respiratory rate can also be determined from the time-dependent series of Q_{TOT}(t), and minute volume can be determined from knowledge of the respiratory rate and tidal volume.

To obtain Q_{TOT} using equation 37, step 303, there needs to be entrainment of air gas flow by the patient when FiO2 is determined, and so when FiO2 is measured, the flow rate of the apparatus gas flow 11 would preferably be below the inspiratory demand of the patient (inspiratory demand herein can refer to instantaneous inspiratory demand and/or or peak inspiratory demand unless otherwise stated). This means that the apparatus gas flow 11 should at least temporarily be below inspiratory demand of the patient (e.g. instantaneous inspiratory demand or peak inspiratory demand). For example, the flow rate is below inspiratory demand for at least part of inspiration. For example, for some of inspiration, the apparatus flow 11 is at a first flow rate which is at or above inspiratory demand, and for some of inspiration the apparatus gas flow 11 is at a second or subsequent flow rate, each of which are below inspiratory demand. While the apparatus flow 11 could be permanently below inspiratory demand (flow rate) of the patient, this would be undesirable as the apparatus gas flow 11 would be sub therapeutic/would not provide the desired respiratory support. Therefore, in one option, the flow rate of the apparatus gas flow 11 is reduced below inspiratory demand temporarily when the FiO2 measurement is taken. In one option, the flow rate of the apparatus gas flow could be varied, in step 300, in any suitable manner. For example it might be varied to dip below the inspiratory demand flow rate periodically as measurement is taken. Preferably, the time at which the flow rate of the apparatus gas flow dips below the inspiratory demand flow rate is kept as short as possible, so as to minimise interruption to respiratory support. Time varying flow rate/gas fraction can be at a frequency higher than the breath frequency of the patient. Time varying flow rate/gas fraction can be at a frequency lower than the breath frequency of the patient. Q_{TOT} indicates inspiratory demand flow rate, so by determining Q_{TOT} inspiratory demand can be determined, which assists in delivering an apparatus gas flow flow rate below inspiratory demand, where required. That is, inspiratory deman flow rate can be determined based on: the target gas input, time-varying flow rate, gas proportion.

Other respiratory parameters could be determined instead or additionally as previously noted.

3. Determining Exhaled Gas Flow Flow Rate and/or Respiratory Parameters

During the Expiratory Cycle when the Patient Mouth is Closed Referring to FIG. 4, in one embodiment, a patient expiratory flow rate is determined from a combination of state parameters, and then from that optionally one or more of the respiratory parameters are found. The method of this embodiment is carried out when the patient's mouth is closed, during patient expiration. The patient's mouth closed can be determined in any suitable way. For example, a person might look at a patient and see their mouth is closed, and provide input to an apparatus of the mouth state being closed. Other options are possible. The mouth being closed is a special case of the constant k=0, as noted above. In this case k is constant as the mouth is closed all the time. In the more general case, k might vary over time as the mouth “openness” varies over time. In this case, the embodiment later relating to the mouth and nose being open is relevant, and k can become depending on t, that is k(t). In that event, k it a time variable constant, that is a constant in a mathematical equation sense, but which can vary so is not a constant value over time. It should be noted that even in the open mouth situation, k might not be dependent on time, so both time dependent and non-time dependent k is possible. The constant k will be described further in relation to embodiments where the mouth is not closed.

The method is carried out on an apparatus, such as described below. The full derivation of the equations used by the method is set out in the derivations section below.

This embodiment utilises an apparatus gas flow 11 that has a time-varying flow rate or a time-varying oxygen fraction. More generally another gas (e.g. CO2, N2 or the like) could provide a time-varying gas fraction marker in the apparatus gas flow 11, but O2 will be used here as an exemplary option. And the gas fraction could more generally be any gas proportion. The time-varying flow rate and time-varying oxygen fraction options will be described separately.

### 3.1 Determining Respiratory Parameters Using Time-Varying Flow Rate or O2 Fraction of Apparatus Gas Flow

In summary, the exhaled gas flow (patient expiration) 13 flow rate Q_{E} of the patient is found using:

$${Q}_{RE} = {Q}_{O} ( t ) \frac{{F}_{O} ( t ) - {F}_{m} ( t )}{{F}_{m} ( t ) - {F}_{E} ( t )} \qquad ( 41 )$$

where
Q_{E} is the expiratory flow rate (exhaled gas-flow flow rate)
and the state parameters are
Qₒ is the flow rate of the apparatus gas flow 11
F_{O} is the volume fraction of the gas (e.g. O2, N2 or tracer gas) component in the apparatus gas flow 11 coming from the respiratory apparatus
Fₘ is the volume fraction of the gas (e.g. O2, CO2, N2 or tracer gas) component in the composite gas outflow 15 from the patient.
F_{E} is volume fraction of the gas (e.g. CO2, O2, N2 or tracer gas) in the exhaled patient gas flow 13 (volume fraction of expired gas). It's determination is set out below for both varying flow rate and varying gas fraction apparatus gas flows.

Once Q_{E} is calculated it can be used to find the tidal volume (a respiratory parameter) by integrating Q_{E} over the expiration cycle. The tidal volume can be defined as follows.

$${V}_{Tidal} ( t ) = \int {Q}_{E} ( t ) dt \qquad ( 42 )$$

In addition other respiratory parameters can be found as follows.
Minute ventilation
Respiratory rate
Apnoea
Airway patency

Referring to the flowchart in FIG. 4, the method will now be described in further detail.

Qₒ, F_{O} are known operating parameters of the apparatus/apparatus gas flow 11, step 404. This means, to determine Q_{E} from equation 41, Fₘ and F_{E} need to be determined. This will be explained.

### 3.1.1 Determining Fₘ

An apparatus gas flow 11 is generated and provided by the respiratory apparatus 10 towards the patient via a patient interface, step 400.

During the expiration cycle of the patient, step 401, Fₘ is measured, step 402, by a suitable sensor 14, for example an oxygen or CO2 fraction sensor. The sensor may be arranged to sense proximate the nares of the patient and/or in nares of the patient. For example, the oxygen fraction sensor 14 could be on the cannula 51 providing the apparatus gas flow 11. The oxygen sensor 14 measures the oxygen fraction of the composite gas flow 17 leaving (being exhaled) by the patient. The measurement could be taken at time intervals (e.g. continuously or at least periodically/discretely) over time during an expiration cycle. For example, the measurement can be taken at Fₘ (t), Fₘ (t+Δt) etc. This provides multiple measurements, step 402, along with Qₒ, F_{O}, which are known, step 404, which can result (once F_{E} is also found, see next) in a corresponding Q_{E} (t), (t+Δt) etc. values calculated at those time intervals using the equation 41, step 405.

### 3.1.2 Determining F_{E}

During the expiration cycle of the patient, step 401, F_{E} is also determined, step 403, as follows. A full explanation and derivation will be described in the derivations section. Determining F_{E} is described in PCT/IB2021/052062, U.S. application 62/989,081 (from which PCT/IB2021/052062 claims priority), both of which are incorporated herein in their entirety. F_{E} Could be a measure of CO2 fraction (FECO2) or O2 fraction (FEO2), both of which are covered herein and described in the incorporated applications.

The present embodiments relate to a non-sealing respiratory apparatus 10 that provides apparatus gas flow 11 to a patient. The non-sealing apparatus means some of the apparatus gas flow 11 does not enter the lower airways of the patient, but rather “leaks” (leak gas flow 12) to ambient. This results in a “Composite gas outflow” (also termed “total gas outflow”), which is the leak gas flow 12 combined with the exhaled gas flow 13. This means that when measuring Fₘ (that is, a fraction of oxygen or CO2 or other gas from the patient), it is the gas fraction of the composite gas flow 15 that is measured, not the gas fraction F_{E} of the exhaled gas flow 13. To find the fraction of gas of the exhaled gas flow F_{E}, the method of PCT/IB2021/052062, U.S. application 62/989,081 can be used, also as explained herein. The embodiments provide an apparatus and method to determine an actual exhaled gas flow 13 parameter F_{E} of a desired gas component by measuring a parameter of the gas component in a composite gas outflow 15 at or near (“proximate”) the patient, and taking into account the effect of the leak gas flow 12 in the measure of the parameter and adjusting the measure accordingly (or otherwise using the measure and other information) to determine the parameter of the desired gas component in the actual exhaled gas flow 13 from the patient. The apparatus gas flow can be varied with a signature to assist to determine the parameter. Note, the composite gas outflow 15 can comprise other gases too (in addition to CO2 and O2)—e.g. those present in ambient air. The present embodiments described work in the presence of such additional gases.

Finding F_{E} will now be described. The respiratory apparatus 10 can comprise a flow source that is able to provide apparatus gas flow to the patient. The apparatus is described further below with reference to FIG. 8. The apparatus 10 provides a time-varying apparatus gas flow, such that a time-varying parameter of the apparatus gas flow varies over time. This provides a signature that can be used to determine a gas parameter of actual exhaled gas flow 13. As possible examples, the time-varying parameter of the apparatus gas flow might be flow rate, or a gas proportion (such as gas fraction (e.g. O2 fraction) and/or gas partial pressure (e.g. O2 partial pressure)). The time varying flow rate/gas proportion can be at a frequency higher than the breathing frequency of the patient. The time varying flow rate/gas proportion can be at a frequency lower than the breathing frequency of the patient. This embodiment uses CO2 sensing of the patient, but alternatively O2, N2 tracer, anaesthetic or other gas sensing could be used.

### 3.1.2.1 Finding F_{E} Using Time-Varying Flow Rate

In one embodiment, the apparatus gas flow flow rate is time varied in a suitable manner, as described elsewhere herein.

Then, F_{E} can be determined using one of the following equations, the derivations of which are set out later.

For determining (general case) exhaled gas fraction F_{E} the following is used:

$${F}_{E} ( t ) \approx \frac{\begin{matrix}{Q}_{o} ( t ) {F}_{m} ( t + Δ t ) ( {F}_{o} ( t ) - {F}_{m} ( t ) ) - \\ {Q}_{o} ( t + Δ t ) {F}_{m} ( t ) ( {F}_{o} ( t + Δ t ) - {F}_{m} ( t + Δ t ) )\end{matrix}}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

For determining exhaled CO2 fraction F_{E} the following is used, which is derived from the above:

$${F}_{E} ( t ) ∼ {F}_{m} ( t + Δ t ) {F}_{m} ( t ) \cdot \frac{{Q}_{o} ( t + Δ6 t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

F_{E} can be re-designated FECO2 where it is CO2 that is being determined.

### 3.1.2.2 Finding F_{E} Using Time-Varying O2 Fraction

In one embodiment, the apparatus gas flow gas proportion (such as O2 fraction) is time varied in a suitable manner, as described elsewhere herein.

Then, for determining exhaled O2 fraction F_{E} the following is used:

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

F_{E} can be re-designated FEO2 where it is O2 that is being determined.

### 3.1.3 Q_{E}, Tidal Volume and Other Respiratory Parameters

Once Fₘ and F_{E} have been determined (measurement and equation 16 (for time-varying flow rate) or 17 (for time-varying oxygen fraction) respectively) as above, Q_{E} can be found from equation 41, step 405.

Then, once Q_{E} is calculated, it can be used to find the tidal volume (a respiratory parameter) by integrating Q_{E} over the inspiration cycle using equation 42 step 406.

Other respiratory parameters could be determined instead or additionally as previously noted.

4. Determining Expiratory Flow Rate and/or State Parameters During the Expiratory Cycle when the Patient Mouth is Open.

Referring to FIG. 5, in one embodiment, a patient expiratory flow rate is determined from a combination of state parameters, and then from that optionally one or more of the respiratory parameters are found.

The method of this embodiment is carried out when the patient's mouth is open to some degree, during patient expiration.

The method is carried out using an apparatus, such as described below. The full derivation of the equations used by the method is set out in the derivations section below.

This embodiment utilises an apparatus gas flow that has a varying flow rate or a varying oxygen fraction. Each option will be described separately. It is possible for a further embodiment where both the flow rate and the Oxygen fraction are varied.

More generally another gas could provide a time-varying gas fraction marker in the apparatus gas flow 11, but O2 will be used here as an exemplary option. And the gas fraction could more generally be any gas proportion. The time-varying flow rate and time-varying oxygen fraction options will be described separately.

In summary, the exhaled gas flow (patient expiration) 13 flow rate Q_{E} of the patient is found using:

$${Q}_{E} ( t ) = k ( t ) {Q}_{o} ( t ) \frac{{F}_{o} ( t ) - {F}_{m} ( t )}{{F}_{m} ( t ) - {F}_{E} ( t )} \qquad ( 30 )$$

where

Q_{E} is the expiratory flow rate (patient exhaled gas-flow flow rate)

and the state parameters are

k is the proportion of the apparatus gas flow 11 that comes out through the mouth (and (1−k) is the fraction that goes through the nose). It is a value between 0 and 1, inclusive. It is assumed that substantially all patient expiration exits through the mouth when the mouth is open.

F_{O} is the volume fraction of the gas (e.g. O2, N2 or tracer gas) component in the apparatus gas flow 11 coming from the respiratory apparatus

Fₘ is the volume fraction of the gas (e.g. O2, CO2, N2 or tracer gas) component in the composite gas outflow 15 from the patient.

F_{E} is volume fraction of the gas (e.g. CO2, O2, N2 or tracer gas) in the exhaled patient gas flow 13 (volume fraction of expired gas) and can be determined from: In this case k is constant. In the more general case, k might vary over time as the mouth “openness” varies over time. In that event, k is a time variable constant, that is a constant in a mathematical equation sense, but which can vary so is not a constant value over time. It should be noted that even in the open mouth situation, k might not be dependent on time, so both time dependent and non-time dependent k is possible. It will be appreciated that any reference to k could be k(t) or vice versa unless context dictates otherwise.

If using varying flow rate for apparatus gas flow (for O2 fraction exhaled)

$${F}_{E} ( t ) \approx \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

If using varying flow rate for apparatus gas flow (for CO2 fraction exhaled)

$${F}_{E} ( t ) \approx {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

### Or

If using varying oxygen fraction for apparatus gas flow (for O2 fraction exhaled)

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

Equation 30 is similar to equation 41 except that equation 30 has a k(t) term, whereas equation 41 has a (1−k(t)) term. This is because in equation 41, the mouth is closed, so the sensor 14 senses at the nose. In equation 30, the mouth is open and so the sensor 14 senses at the mouth.

Once Q_{E} is calculated it can be used to find the tidal volume (a respiratory parameter) by integrating Q_{E} over the inspiration cycle. The tidal volume can be defined as follows.

$${V}_{Tidal} ( t ) = \int {Q}_{E} ( t ) dt \qquad ( 42 )$$

In addition other respiratory parameters can be found as follows.
Minute ventilation
Respiratory rate
Apnoea
Airway patency

Referring to the flowchart in FIG. 5, the method will now be described in further detail.

Qₒ,F_{O} are known operating parameters of the apparatus/apparatus gas flow 11, step 404. This means, to determine Q_{E} from equation 30, Fₘ, F_{E} and k need to be determined. An apparatus gas flow 11 is generated and provided by the respiratory apparatus 10 towards the patient via a patient interface 51, step 400. During the expiration cycle, step 401, Fₘ and F_{E} can be found as per the previous embodiment, step 402, 403. In effect, Q_{E}, step 405, and tidal volume (or other respiratory parameters), step 406, can be found in the same manner as for the previous embodiment (closed mouth, during expiration cycle), except now k is used as well.

Determination of K, step 500, will now be described.

4.1 Determining k or k(t)

This involves determining the proportion k or more generally k(t) of apparatus gas flow 11 that exits the mouth.

This is a value between 0 and 1 and is the proportion of delivered gases (apparatus gas flow 11) going through the mouth. If the mouth is closed, k=0. If the mouth is open, some proportion k(t) of the apparatus gas flow goes through the mouth, and some proportion (1−k(t)) goes through the nose. As noted above, k might or might not be time variable and any reference to k could be k(t) and vice versa.

In the case where the mouth is closed (k=0), the equations are based on Fₘ being measured through a sensor 14 sensing at the nose. In the case of the mouth being open (k between 0 and 1), the equations are based on Fₘ being measured at the mouth. “At” in this context means, near, at, in proximity, vicinity or any other term indicating the location of the sensor's sensing portion/input being placed where it can suitably measure the relevant gas flow.

It will be appreciated that while k has been defined with reference to the proportion of apparatus gas flow coming out of the mouth, a mathematically and/or physically analogous constant could be defined, for example with reference to the proportion coming out the nose. Such variants will be understood by those skilled in the art to be equivalent. Therefore the k proportion can more generally be considered a proportion (e.g. volume fraction) of gas flow through the mouth and/or nose

Determination of the proportion k at a particular sensor location can be made by various methods including:
1. Administer flow through one nostril of a sealed interface and measure the flow out the other nostril (Qm) (this could be done using a flow meter or a pressure sensor upstream of a known pressure plate generating a known resistance to ambient). For example, k=(Qₒ-Qₘ)/Qₒ.
2. Deliver flow through a nasal interface sealed at the nostrils so that all delivered flow exits the oral cavity.
3. In a special case for k=0 (mouth closed), observation of oxygen trace throughout a breath cycle. If distinct dips of inspiration and expiration are visible during high flow then the mouth is closed (k=0).

### 4.2 Q_{E}, Tidal Volume and Other Respiratory Parameters

Once Fₘ, F_{E} (measurement and equation 16 or 17 respectively—steps 402, 403) and k (from one of the methods above), step 500, have been determined as above, Q_{E} can be found from equation 30, step 405.

Then, once Q_{E} is calculated, it can be used to find the tidal volume (a respiratory parameter) by integrating Q_{E} over the expiration cycle using equation 42 step 406.

Other respiratory parameters could be determined instead or additionally as previously noted.

### 5. Combined Methods

In another embodiment, several embodiments could be available for use, depending on the state of the patient's mouth and/or the desire to do the method during inspiration or expiration.

In one embodiment, all the methods described above are available for use/implemented in an apparatus 10 as described herein, and the method used is dependent on the state of the patient's mouth and/or the part of the respiratory cycle.

FIG. 6 shows how the methods are combined. In such an embodiment, the method used for determining a respiratory parameter is based on whether the patient's mouth is open or closed, step 61 and whether the patient is inspiring or expiring (respiratory phase), step 60. These states (including respiratory phase) can be determined using any suitable manner. For example a CO2 trace, ECG or respiratory band could be used, as just some non-limiting examples. If the mouth is closed, step 62, and the patient is inspiring, step 64, the method of FIG. 3 is used, step 67. If the mouth is closed, step 62, and the patient expiring, step 64, the method of FIG. 4 is used, step 66. If the mouth is open, step 62 and the patient is expiring, step 63, the method of FIG. 5 is used, step 65. Otherwise if the mouth is open the breath is monitored until expiration is detected. Any one or combination of the methods can be used as required depending on the patient state.

FIG. 7 shows a non-limiting example, which is one possible implementation of the combined method. The apparatus gas flow 11 at the sensor location (be it mouth or nose) is determined and k is obtained, step 70. From this it can be determined if the mouth is open or closed.

If the mouth is closed, and k=0, it is then determined if the patient is expiring or inspiring. When the patient is inspiring and mouth closed, path 71 is taken. The gas fraction at the patient sensor 14 at the nose is measured, step 71A, in this case FiO2, measured at time t with apparatus gas flow Q₀1, step 71B. Next FiO2 is measured at time (t+Δt) with apparatus gas flow Qₒ2, step 71C. Q_{TOT} is determined based on FiO₂ measurements, step 71D. This can be integrated between the time point to determine tidal flow. This process can be repeated for subsequent time points, step 75.

When the patient is expiring path 73 or 74 is taken, irrespective of whether the mouth is opened or closed. However, if the mouth is open, k is determined at step 70. Then gas fractions are measured, step 72, and the flow rate or the O2 proportion is oscillated, path 73 or path 74. In the case of oscillating oxygen, Step 74A, the gas fraction at the patient sensor 14 at the nose and/or mouth is measured, step 74B in this case FₘCO₂/F_{mO2}, measured at time t with apparatus O₂ fraction F_{O2_1}. Next FₘCO₂/F_{mO2} is measured at time (t+Δt) with O₂ fraction F_{O2_2}, step 74C. Q_{E} is determined, step 74D, based on FeCO₂/FeO₂ determination. This can be integrated between the time point to determine tidal flow. This process can be repeated for subsequent time points, step 75.

In the case of oscillating flow rate, step 73A, the gas fraction at the patient sensor 14 at the mouth and/or nose is measured, step 73B, in this case FₘCO₂/F_{mO2}, measured at time t with flow Q_{O1}. Next FₘCO₂/FmO₂ is measured, step 73C, at time (t+Δt) with flow Q_{O2}. Q_{E} is determined, step 73D, based on FeCO₂/FeO₂ determination. This can be integrated between the time point to determine tidal flow.

This process can be repeated for subsequent time points, step 75.

### 6. Apparatus for Providing Gas Flow and Determining Respiratory Parameters

An apparatus and method for determining the respiratory parameter as described above will be described with reference to the apparatus of FIG. 8 and the methods described with reference to previous FIGS. 3 to 7. This apparatus can also be used for other embodiments described herein. The apparatus of FIG. 8 is a more detailed explanation of the apparatus of FIG. 1B.

The respiratory apparatus 10 can provide the apparatus gas flow 11 used in the methods described herein. It can also have the sensors 14 to make various measurements for the methods. Various implementations of sensors are possible, and while a sensor can be placed anywhere suitable, when referencing position relative to mouth and/or nose, that is an indication of where the sensing portion is positioned. For example, in a sensor with a sampling conduit, the sampling conduit (sensing portion) is near mouth and/or nose, even if the sensor or parts of sensor are elsewhere. Other implementations of sensor will be known to those skilled in the art too. The controller of the apparatus can carry out some or all of one or more of the methods. Optionally, other external apparatus could be used alternatively or in addition to the respiratory apparatus to carry out some or all or one or more of the methods.

FIG. 8 shows a respiratory apparatus 10 for providing flow therapy or other therapy (respiratory support) to a patient. The apparatus is configured for delivering a time-varying apparatus gas flow 11 and carrying out the processing for determining the desired respiratory parameters (e.g. tidal volume, minute ventilation, respiratory rate, apnoea, airway patency or the like). The apparatus 10 could be an integrated or a separate component based arrangement, generally shown in the dotted box in FIG. 8. In some configurations, the apparatus could be a modular arrangement of components. As such, the apparatus could be referred to as a “system” but the terms can be used interchangeably without limitation. Hereinafter it will be referred to as an apparatus, but this should not be considered limiting. The apparatus may be used for any suitable purpose including preoxygenation during an anaesthetic procedure, during an anaesthetic procedure, high flow therapy, ventilation, whilst treating patients in respiratory distress, treating patients with obstructive sleep apnoea or anywhere else where monitoring of an aspect of patient breathing is required.

The apparatus comprises a flow source 50 for providing a high flow gas 31 such as oxygen, or a mix of oxygen and one or more other gases. Alternatively, the apparatus can have a connection for coupling to a flow source. As such, the flow source might be considered to form part of the apparatus or be separate to it, depending on context, or even part of the flow source forms part of the apparatus, and part of the flow source fall outside the apparatus.

The flow source could be an in-wall supply of oxygen, a tank of oxygen 50A, a tank of other gas and/or a high flow respiratory apparatus with a blower/flow generator 50B. FIG. 8 shows a flow source 50 with a flow generator 50B, with an optional air inlet 50C and optional connection to an O2 source (such as tank or O2 generator) 50A via a shut off valve and/or regulator and/or other gas flow control 50D, but this is just one option. The description from here can refer to either embodiment. The flow source could be one or a combination of a flow generator, O2 source, air source as described. The flow source 50 is shown as part of the apparatus 10, although in the case of an external oxygen tank or in-wall source, it may be considered a separate component, in which case the apparatus has a connection port to connect to such flow source. The flow source provides a (preferably high) flow of gas that can be delivered to a patient via a delivery conduit, and patient interface 51. Depending on the end-use, the patient interface 51 may be an unsealed (also termed “non-sealing”) interface (for example when used in high flow therapy) such as a nasal interface (cannula), or a sealed interface (for example when used in CPAP) such as a nasal mask, full face mask, or nasal pillows.

The time-varying flow rate embodiment can be used with a non-sealing patient interface. A time-varying flow rate gas flow is not passed to or through a cavity external to the patient, so e.g. is preferably passed through a non-sealing nasal cannula. An external cavity might introduce a low pass filter that might attenuate a time-varying flow rate signature. The time-varying fraction embodiment can be used with a sealed patient interface or a non-sealing patient interface. The patient interface 51 is preferably a non-sealing patient interface which would for example help to prevent barotrauma (e.g. tissue damage to the lungs or other organs due to a difference in pressure relative to the atmosphere). The patient interface may be a nasal interface (cannula) with a manifold and nasal prongs, and/or a face mask, and/or a nasal pillows mask, and/or a nasal mask, and/or a tracheostomy interface, or any other suitable type of patient interface. The flow source could provide a therapeutic gas flow rate of between, e.g. about 0.5 litres/min and about 375 litres/min, or any range within that range, or even ranges with higher or lower limits.

The time-varying apparatus gas flow may have an therapeutic time-varying (e.g. oscillating) flow rate and the controller controls a gas flow modulator to provide the therapeutic time-varying apparatus gas flow with an oscillating flow rate of: about 375 litres/min to about 0 litres/min, or preferably of about 240 litres/min to about 7.5 litres/min, or more preferably of about 120 litres/min to about 15 litres/min, and/or the oscillating flow rate has one or more frequencies of about 0.1 Hz to about 200 Hz, and preferably about 0.1 Hz to about 6 Hz, and more preferably about 0.5 Hz to about 4 Hz, and more preferably 0.6 Hz to 3 Hz. The gas flow modulator might be the flow source (where that could be a flow generator, O2 source, ambient air or the like as previously discussed) and/or a valve or other device to modulate or otherwise vary parameters (e.g. flow rate, gas proportion) of a gas flow.

The oscillating flow rate may comprise a therapeutic flow rate component, wherein the therapeutic flow rate is about 375 litres/min to about 0 litres/min, or about 150 litres/min to about 0 litres/min, or is preferably about 120 litres/min to about 15 litres/min, or is more preferably about 90 litres/min to about 30 litres/min.

The oscillating flow rate may comprise a therapeutic gas flow component, wherein the constant (e.g. bias/base) flow rate component of the therapeutic gas flow is about 0.5 litres/min to about 25 litres/min.

The oscillating flow rate may comprise a therapeutic flow rate component, wherein the therapeutic flow rate is about 0.2 litres/min per patient kilogram to about 2.5 litres/min per patient kilogram; and preferably is about 0.25 litres/min per patient kilogram to about 1.75 litres/min per patient kilogram; and more preferably is about 0.3 litres/min per patient kilogram to about 1.25 litres/min per patient kilogram or about 1.5 litres/min per patient kilogram; and more preferably is about 0.4 litres/min per patient kilogram to about 0.8 litres/min per patient kilogram.

The one or more components of the time-varying (e.g. oscillating) gas flow may have one or more frequencies of about 0.3 Hz to about 4 Hz.

The oscillating flow rate may comprise at least one time-varying flow rate component, wherein each oscillating flow rate is about 0.05 litres/min per patient kilogram to 2 litres/min per patient kilogram; and preferably is about 0.05 litres/min per patient kilogram to about 0.5 litres/min per patient kilogram; and preferably about 0.12 litres/min per patient kilogram to about 0.4 litres/min per patient kilogram; and more preferably about 0.12 litres/min per patient kilogram to about 0.35 litres/min per patient kilogram. Alternatively, the oscillating flow rate may comprise at least one time-varying flow rate component, wherein each oscillating flow rate is in the range of 0.05 litres/min per patient kilogram to 2 litres/min per patient kilogram; and preferably in the range of 0.1 litres/min per patient kilogram to 1 litres/min per patient kilogram; and more preferably in the range of 0.2 litres/min per patient kilogram to 0.8 litres/min per patient kilogram.

The above are examples of therapeutic time-varying flow rates. Signature flow rates could be provided also and could be lower, the same or higher than therapeutic flow rates. Signature flow rate frequencies could be lower, the same or higher than therapeutic flow rate frequencies (when time-varying). In some embodiments, the signature flow rate has a higher frequency than the therapeutic flow rate.

As an example, the signature flow rates can step between a first flow rate and second flow rate, one or both of which can fall in the range of about 0 LPM to 70 LPM. The maximum signature flow rate could be the therapeutic flow rate. The signature flow rate could be combined with the therapeutic flow rate (e.g. added) or it could form part or all of the therapeutic flow rate—that is, the therapeutic flow rate itself could be the signature flow rate. In some embodiments, the signature flow rate can be related to the therapeutic flow rate as a percentage. For example, the signature time-varying flow rate (adults) is in the range of:
about 0% to about 200% of the therapeutic flow rate,
about 0% to 100% of the therapeutic flow rate,
about 100% to 200% of the therapeutic flow rate, or
about 50% to 150% of the therapeutic flow rate, and/or is in the range of
about 0-140 LPM,
about 0-70 LPM,
about 70-140 LPM,
about 40-100 LPM, or
about 20-60 LPM

These are not limiting flow rates, and it should also be noted that the signature flow rates could be negative, but when combined with the therapeutic flow rates create a total flow rate that is positive.

In some embodiments, the therapeutic flow rates might also serve as signature flow rates. That is, they perform a dual purpose.

The above are just example, and other types of time-varying flow rates could be provided and the controller controls a gas flow modulator to provide the time varying apparatus gas flow with the time-varying flow rate. The apparatus can have knowledge of the time-varying flow rate, and/or can measure the time-varying flow rate provided, e.g. by a flow sensor, e.g. 53A, 53B, 53C, 53D.

A humidifier 52 can optionally be provided between the flow source 50 and the patient to provide humidification of the delivered gas. This could be a humidifier integrated with the flow source 50 to form an integrated apparatus (see dotted lines) or separate but attachable to the flow source 50. Alternatively, the humidifier 52 could be a standalone humidifier with a chamber and base, where the humidifier is coupled to the flow source 50 via conduits or other suitable means. One or more sensors 53A, 53B, 53C, 53D such as flow rate, oxygen fraction CO2 or other gas fraction, full or partial pressure, humidity, temperature or other sensors can be placed throughout the apparatus and/or at, on or near the patient 14. Alternatively, or additionally, sensors from which such parameters can be derived could be used. In addition, or alternatively, the sensors 53A-53D can be one or more physiological sensors for sensing patient physiological parameters such as, heart rate, oxygen saturation (e.g. pulse oximeter sensor 54E), partial pressure of oxygen in the blood, respiratory rate, partial pressure of O2 and/or CO2 in the blood. Alternatively, or additionally, sensors from which such parameters can be derived could be used. Other patient sensors could comprise EEG sensors, torso bands to detect breathing, and any other suitable sensors, which among other things may sense if a patient is expiring or inspiring (respiratory phase/state). In some configurations the humidifier may be optional, or it may be preferred due to the advantages of humidified gases helping to maintain the condition of the airways. Humidification is preferably used with high flow gas flows to increase patient comfort, compliance, support and and/or safety. One or more of the sensors might form part of the apparatus, or be external thereto, with the apparatus having inputs for any external sensors.

A sensor 14 for measuring the gas parameter (of the target gas) of the patient composite gas inflow and outflow 15 is provided. The sensor could sense at the mouth and/or nose. It can be placed anywhere suitable, which might at the mouth and/or nose, or distil from it with a sensing portion at the mouth and/or nose. That is, depending on the target gas e.g. oxygen, carbon dioxide, nitrogen, helium and/or an anaesthetic agent such as sevoflurane, a sensor is chosen to sense that gas in the composite gas outflow. The sensor e.g. can be a mainstream or side stream sensor, and can be placed proximate (in, on, near) the nose and/or mouth. Other positions are possible. The time-varying flow rate embodiment can work with one gas parameter sensor—e.g. it can work when one gas parameter (e.g. fraction CO2, or fraction O2) is measured. In the time-varying flow rate embodiment, it is not essential to measure more than one gas parameter to obtain the target parameter (e.g. it is not necessary to measure fraction CO2 and fraction O2 to implement the embodiment herein).

The output from the sensors is sent to a controller to assist control of the apparatus, including among other things, to vary gas flow or to provide for the determination and display of a respiratory parameter in the case of sensor 14. Alternatively, or additionally, input could come from a user. The controller is coupled to the flow source, humidifier and sensors. It controls these and other aspects of the apparatus to be described below. The controller can operate the flow source to provide the delivered flow of gas. It can also operate the gas flow modulator(s) (including the flow source) to control the flow, pressure, volume and/or other parameters of gas provided by the flow source based on feedback from sensors, or optionally without feedback (e.g. using default settings or user input). The controller can also control any other suitable parameters of the flow source to meet oxygenation requirements and/or CO₂ removal. The controller 19 can also control the humidifier 52 based on feed-back from the sensors 53A-53D, 14. Using input from the sensors, the controller can determine oxygenation requirements and provide information to a medical professional (who may control the components of the respiratory apparatus to provide the desired therapy, e.g. flow rate, O₂ fraction, humidity, etc.) and/or control parameters of the flow source, gas flow modulator(s) and/or humidifier as required. Alternatively, the embodiments could be provided as a standalone monitoring apparatus, independent of a respiratory apparatus that provides information to a medical professional and/or communicates with and/or controls components of the respiratory apparatus to provide a desired therapy or respiratory support. The medical professional can then control the respiratory apparatus to provide the desired therapy. Accordingly, the controller may not always determine oxygenation requirements and/or control parameters of the apparatus.

The controller 19 is also configured to operate the apparatus so that the apparatus gas flow has a time-varying flow rate that provides therapy and also a signature flow rate as described. It can do this through any suitable means such as controlling flow generator 50B or any other suitable gas modulator. The gas modulator can be used to modulate (that is, varying, modify, adjust or otherwise control parameters of the gas flow). Each gas flow modulator can be provided in the flow source (and the flow source itself can be a gas flow modulator), after the flow source and before the humidifier, after the humidifier, and/or in any other suitable place in the apparatus to modulate gas flow path. It can also operate the gas flow modulator(s) (including the flow source) to control the flow, pressure, volume and/or other parameters of gas provided by the flow source based on feedback from sensors, or optionally without feedback (e.g. using default settings or based on user input). The controller can also control any other suitable parameters of the flow source to meet oxygenation requirements. The gas modulator could be, for example, anything described in WO2017/187390 or US20210052844 which are incorporated herein by reference in their entirety.

The controller can then measure the composite gas outflow and determine the gas and/or respiratory parameter using any of the techniques described throughout.

For other embodiments below relating to varying gas proportion, the controller 19 is additionally or alternatively configured to operate the apparatus so that the apparatus gas flow has a time-varying gas proportion (such as O2 fraction or other gas fraction and/or O2 partial pressure or other gas partial pressure) that provides therapy/respiratory support, and also a signature gas proportion (such as gas fraction and/or gas partial pressure) as described. It can do this through any suitable means such as controlling a proportional valve coupled to an O2 source 50A or any other means previously described in other patents. The controller can then measure the composite gas outflow and/or determine (e.g. obtain an estimate of) the gas and/or respiratory parameter using any of the techniques described throughout. In one embodiment, there are two proportional valves that operate 180 degrees out of phase. As one opens, the other closes. One controls O2 fraction in the apparatus gas flow, and the other controls Air fraction in the apparatus gas flow, but together keeping the total gas flow rate of the apparatus gas flow constant. In another alternative, a single proportional valve is used with an impeller/Flow generator where the proportional valve controls an O2 fraction and the impeller controls the flow rate. In some embodiments, the single proportional valve may be used before or after the impeller. Where the single proportional valve is used before the impeller, the proportional valve controls the O2 fraction into the inlet of the impeller along with the ambient air. In some embodiments, more than one proportional valve may be used with an impeller and may be positioned anywhere in the system with respect to the impeller. The controller 19 can control the proportional valve(s) to operate as required to achieve the time-varying gas proportion as described herein.

An input/output interface 54 (such as a display and/or input device) is provided. The input device is for receiving information from a user (e.g. clinician or patient) that can be used for example for determining oxygenation requirements, anaesthetic gas agent, detection, flow rates, gas fractions, partial pressures and/or any other parameter that might be controlled by the apparatus.

The apparatus also comprises a display which can be part of the I/O for displaying the measure of the gas parameter of the exhaled gas flow, as a graph, digital readout or any other suitable means. It can also display a state and/or respiratory parameter, such as tidal volume (either instantaneous or average over number of cycles) and/or patient gas flow. It could display a value or average value or trace.

The apparatus can include one or more communication modules 59 to enable data communication or connection with one or more external devices or servers over a data or communication link or data network, whether wired, wireless or a combination thereof. In one configuration for example, the apparatus can include a wireless data transmitter and/or receiver, or a transceiver 59 to enable the controller 19 to receive data signals in a wireless manner from the operation sensors and/or to control the various components of the system. The transceiver 59 or data transmitter and/or receiver module may have an antenna. In one example, the transceiver may comprise a Wi-Fi modem. Additionally, or alternatively, the data transmitter and/or receiver 59 can deliver data to a remote patient management system (i.e. remote server) 69 or enable remote control of the system. The system can include a wired connection, for example, using cables or wires, to enable the controller 19 to receive data signals from the operation sensors and/or to control the various components of the apparatus 10. The apparatus 10 may comprise one or more wireless communication modules. For example, the apparatus may comprise a cellular communication module such as for example a 3G, 4G or 5G module. The module 59 may be or may comprise a modem that enables the apparatus to communicate with a remote patient management system (not illustrated in the figures) using an appropriate communication network. The remote management system may comprise a single server or multiple servers or multiple computing devices implemented in a cloud computing network. The communication may be two-way communication between the apparatus and a patient management system (e.g. a server) or other remote system. The apparatus 10 may also comprise other wireless communication modules such as for example a Bluetooth module and/or a Wi-Fi module. The Bluetooth and/or WiFi module allow the apparatus to wirelessly send information to another device such as for example a smartphone or tablet or operate over a LAN (local area network) or Wireless LAN (WLAN). The apparatus may additionally, or alternatively, comprise a Near Field Communication (NFC) module to allow for data transfer and/or data communication.

For example, measured patient breathing parameter data (e.g., inspiratory, expiratory, and/or total respiratory time ratios) may be communicated to a remote patient management system (i.e. a remote server). The remote patient management system may be a single server or a network of servers or a cloud computing system or other suitable architecture for operating a remote patient management system. The remote patient management system (i.e. remote server) further includes memory for storing received data and various software applications or services that are executed to perform multiple functions. Then, for example, the remote patient management system (i.e. remote server) may communicate information or instructions to the system 10 at least in part dependent on the data received. For example, the nature of the data received may trigger the remote server (or a software application running on the remote server) to communicate an alert, alarm, or notification to the system 10.

The remote patient management system may further store the received data for access by an authorized party such as a clinician or the patient or another authorized party. The remote patient management system may further be configured to generate reports in response to a request from an authorized party, and the breathing parameter data e.g. inspiratory, expiratory and/or total respiratory time ratios may be included into the generated reports. The reports may further comprise other patient breathing parameters e.g. respiratory rate or SpO2 and/or device parameters e.g. flow rate, humidity level.

As previously noted, the controller 19 implements one or more of the methods described herein. Depending on the method embodiment used, one or more of the following state parameters are acquired using the apparatus or other means, as indicated.
a) The state of the mouth, which might be a binary open/closed, or some parameter indicating the proportion of gas flow (e.g. volume proportion) from the apparatus gas flow exiting the mouth with respect to the nose. E.g. k
b) Flow rate of the gas flow from the respiratory apparatus. E.g. Qₒ
c) Gas fraction (which includes any equivalent gas proportion) of the gas flow from the respiratory apparatus. E.g. O2 fraction but could be any other gas proportion where suitable, e.g. N2 or tracer gas F_{O}, but could be any other gas proportion where suitable, e.g. N2 or tracer gas
d) Gas fraction of gas flow into or out of the patient (depending if measured in inhalation or exhalation). E.g. O2 fraction Fₘ but could be any other gas proportion where suitable, e.g. CO2, N2 or tracer gas
e) Gas fraction of exhaled gas flow by the patient. F_{E} could be any gas proportion where suitable, e.g. O2, CO2, N2 or tracer gas
f) Gas fraction and flow rate of entrained (ambient) gas. Qₑₙₜ, Fₑₙₜ (note, can also be referred to as F_{entrained} which will be used interchangeably herein). But could be any gas proportion where suitable, e.g. O2, CO2, N2 or tracer gas
g) Flow rate of patient gas flow (which can be a respiratory parameter as well as a state parameter) being the:
   a. flow rate Q_{E} of exhaled gas flow 13 (that is—gas flow expired by the patient), and/or
   b. flow rate Qₜₒₜ of composite gas inflow 17 (that is—composite gas flow inspired by the patient). Note, Q_{TOT} also indicates the inspiratory demand flow rate of the patient

Above, the gas fraction could be the O2, CO2 or other gas fraction. Partial pressure could be used instead of gas fraction, and that will be understood as interchangeable by a skilled person throughout this specification.

The controller uses one or more of the state parameters as per one or more of the methods described herein to determine Q_{E} or Qₜₒₜ and then tidal volume and/or one or more of the other respiratory parameters as described herein.

It will be appreciated that the embodiments above are particular examples of using one or more state parameters to determine one or more respiratory parameters. In each case there could be a gas such as O2, CO2, N2 other trace gas or anaesthetic that is used as a marker from which to make determinations. Any reference to a particular gas above is by way of example only and should not be so limited where alternatives are readily used.

### 7. Derivations

The derivations for the various equations used are set out in this section.

The equations refer to a constant k. This is a value between 0 and 1 and is the proportion of delivered gases (apparatus gas flow 11) going through the mouth. If the mouth is closed, k=0. If the mouth is open, some proportion k of the apparatus gas flow goes through the mouth, and some proportion (1−k) goes through (e.g. comes out of/exits) the nose. K can be time constant. In the more general case, k might vary over time as the mouth “openness” varies over time. In this case, k can become dependent on t, that is k(t). In that event, k it a time variable constant, that is a constant in a mathematical equation sense, but which can vary so is not a constant value over time. It should be noted that even in the open mouth situation, k might not be dependent on time, so both time dependent and non-time dependent k is possible. Reference to k or k(t) should not be considered limiting herein, and either can be used as context allows

In the case where the mouth is closed (k=0), the equations are based on Fₘ being measured through a sensor 14 at the nose. In the case of the mouth being open (k between 0 and 1), the equations are based on Fₘ being measured at the mouth.

7.1 Determining Composite Gas Inflow Flow Rate and/or Respiratory Parameters During the Inspiratory Cycle when the Patient Mouth is Closed.

This embodiment utilises equation 37 below to obtain Q_{TOT}—the flow rate of the (total inhaled) patient gas inflow (composite gas inflow) 17 (apparatus gas flow 11 and entrained air gas flow 16) into the patient

$${Q}_{Tot} ( t ) = ( 1 - k ( t ) ) {Q}_{o} ( t ) [ 1 + \frac{Fo ( t ) - {FiO}_{2} ( t )}{{FiO}_{2} ( t ) - {F}_{entrained} ( t )} ] = {Q}_{o} ( t ) [ 1 + \frac{Fo ( t ) - {FiO}_{2} ( t )}{{FiO}_{2} ( t ) - {F}_{entrained} ( t )} ] \qquad ( 37 )$$

where

$${Q}_{Tot} ( t ) = {Q}_{entrained} ( t ) + {Q}_{o} ( t ) \qquad ( 35 )$$

and the state parameters are:

Qₒ is the flow rate of the apparatus gas flow 11

F_{O} is the volume fraction of the gas (e.g. O2, N2 or tracer gas) component in the apparatus gas flow 11 coming from the respiratory apparatus.

Fₘ is the volume fraction of the gas (e.g. O2, N2 or tracer gas) component in the composite gas inflow (patient inspiratory gas flow) to the patient. As this is during inspiration, Fₘ is FiO2.

F_{entrained} is the volume fraction of the gas (e.g. O2, N2 or tracer gas) component in the entrained air gas flow 16.

k is a value between 0 and 1 and is the proportion of delivered gases (apparatus gas flow 11) going out through the mouth. If the mouth is closed, k=0. In this case k=0 as the mouth is closed, so the (1−k(t)) term becomes 1 and k disappears.

Note reference to O2 herein with reference to above parameters could also be interchanged with N2 or other tracer gas instead.

Once Q_{TOT} is calculated it can be used to find the tidal volume (a respiratory parameter) by integrating Q_{TOT} over the inspiration cycle. The tidal volume can be defined as follows.

$${V}_{Tidal} ( t ) = \int {Q}_{Tot} ( t ) dt \qquad ( 38 )$$

When the mouth is closed, the proportion of apparatus gas flow that exits/enters the mouth is equal to zero.

Thus, if a sensor 14 (e.g. sampling line) is placed next to the patient interface in the patient's nose, then Fₘ (in this case the oxygen fraction (FiO2)) during inspiration can be measured and from this it is possible to determine the sensed parameter (e.g. O2 fraction) of the (instantaneous) flow going into the patient.

$${FiO}_{2} ( t ) = \frac{{F}_{o} ( t ) {Q}_{o} ( t ) + {F}_{entrained} ( t ) {Q}_{entrained} ( t )}{{Q}_{o} ( t ) + {Q}_{entrained} ( t )} \qquad ( 32 )$$

where

FiO2 (t) is the fraction of inspired oxygen, which is the O2 fraction in the composite gas inflow and is measured by sensor 14

Qₒ(t) is the apparatus gas flow flow rate

Fₒ(t) is the volume fraction of the gas (e.g. O2) of the apparatus gas flow (gas coming from the apparatus)

F_{entrained} (t) is the fraction of oxygen in the entrained gas (usually approximately 0.21 in ambient air)

Q_{entrained} (t) is the flow rate of the gas flow entrained from the ambient environment

(Note, measurements of instantaneous FiO2 and calculation of Q_{TOT} are possible when the flow from the high flow system does not meet inspiratory demand at that moment so that there is some entrainment.)

Rearranging yields:

$${Q}_{o} ( t ) + {Q}_{entrained} ( t ) = \frac{{F}_{o} ( t ) {Q}_{o} ( t ) + {F}_{entrained} ( t ) {Q}_{entrained} ( t )}{{FiO}_{2} ( t )} \qquad ( 33 )$$

$${Q}_{entrained} ( t ) ( 1 - \frac{{F}_{entrained} ( t )}{{FiO}_{2} ( t )} ) = \frac{{F}_{o} ( t ) {Q}_{o} ( t )}{{FiO}_{2} ( t )} - {Q}_{o} ( t )$$

$${Q}_{entrained} ( t ) ( 1 - \frac{{F}_{entrained} ( t )}{{FiO}_{2} ( t )} ) = {Q}_{o} ( t ) ( \frac{{F}_{o} ( t )}{{FiO}_{2} ( t )} - 1 )$$

$${Q}_{entrained} ( t ) = {Q}_{o} ( t ) \frac{( \frac{{F}_{o} ( t )}{{FiO}_{2} ( t )} - 1 )}{( 1 - \frac{{F}_{entrained} ( t )}{{FiO}_{2} ( t )} )}$$

$${Q}_{entrained} ( t ) = {Q}_{o} ( t ) \frac{Fo ( t ) - {FiO}_{2} ( t )}{{FiO}_{2} ( t ) - {F}_{entrained} ( t )} \qquad ( 34 )$$

The total flow is given by:

$${Q}_{Tot} ( t ) = {Q}_{entrained} ( t ) + {Q}_{o} ( t ) \qquad ( 35 )$$

Substituting (34) into (35) yields

$${Q}_{Tot} ( t ) = {Q}_{o} ( t ) + {Q}_{o} ( t ) \frac{Fo ( t ) - {FiO}_{2} ( t )}{{FiO}_{2} ( t ) - {F}_{entrained} ( t )} \qquad ( 36 )$$

$${Q}_{Tot} ( t ) = {Q}_{o} ( t ) [ 1 + \frac{Fo ( t ) - {FiO}_{2} ( t )}{{FiO}_{2} ( t ) - {F}_{entrained} ( t )} ] \qquad ( 37 )$$

The tidal volume on inspiration can thus be calculated by:

$${V}_{Tidal} ( t ) = \int {Q}_{Tot} ( t ) dt \qquad ( 38 )$$

$${V}_{Tidal} ( t ) = \int \{ {Q}_{o} ( t ) [ 1 + \frac{Fo ( t ) - {FiO}_{2} ( t )}{{FiO}_{2} ( t ) - {F}_{entrained} ( t )} ] \} dt \qquad ( 39 )$$

where the integral is calculated over one inspiration cycle (where the breath cycle could be determined by various means including looking at when the FiO2 transitions through 1).

As noted above to make measurements of inspiratory flow there should be delivery of less than instantaneous demand. But it is not necessarily desirable to do this throughout the inspiratory cycle because it will reduce the respiratory support (e.g. FiO2) to the patient. So, the flow rate of the apparatus flow can be varied over time (e.g. oscillated or changed between two values) and the measurement taken while the flow rate is low and then use interpolation or similar techniques to derive a continuous measurement of flow and then integrate this continuous flow measurement to find the tidal volume. The frequency of oscillation is optionally greater than the breathing frequency.

Further, the time at the lower flow rate, could be short relative to the time at the higher flow rate to increase the mean FiO2 supplied to the patient. It should be noted that the lower flow rate will need to be greater than zero to allow differentiation of the flows from the high flow system and the entrained air.

Optionally, varying flow rate comprises steps between a higher flow rate that is preferably higher than the patient's inspiratory demand and a lower flow rate that is a non-zero flow rate that is optionally lower than the patient's inspiratory demand.

7.2 Determining Exhaled Gas Flow Flow Rate and/or State Parameters During the Expiratory Cycle when the Patient Mouth is Closed

This embodiment utilises equation 41 below to obtain Q_{E}—the expiratory flow rate (that is, flow rate of the exhaled gas-flow 13)

$${Q}_{E} = ( 1 - k ( t ) ) {Q}_{O} ( t ) \frac{{F}_{O} ( t ) - {F}_{m} ( t )}{{F}_{m} ( t ) - {F}_{E} ( t )} = {Q}_{O} ( t ) \frac{{F}_{O} ( t ) - {F}_{m} ( t )}{{F}_{m} ( t ) - {F}_{E} ( t )} \qquad ( 41 )$$

Qₒ is the flow rate of the apparatus gas flow 11

F_{O} is the volume fraction of the gas (e.g. O2, N2 or tracer gas) component in the apparatus gas flow 11 coming from the respiratory apparatus

Fₘ is the volume fraction of the gas (e.g. O2, CO2, N2 or tracer gas) component in the composite gas outflow 15 from the patient.

F_{E} is volume fraction of the gas (e.g. CO2, O2, N2 or tracer gas) in the exhaled patient gas flow 13 (volume fraction of expired gas) k is a value between 0 and 1 and is the proportion of delivered gases (apparatus gas flow 11) going out through the mouth. If the mouth is closed, k=0. In this case k=0 as the mouth is closed, so the (1−k(t)) term becomes 1 and k disappears.

Once Q_{E} is calculated it can be used to find the tidal volume (a respiratory parameter) by integrating Q_{E} over the expiration cycle. The tidal volume can be defined as follows.

$${V}_{Tidal} ( t ) = \int {Q}_{E} ( t ) dt \qquad ( 42 )$$

An alternative approach to solving the issue of needing to keep apparatus gas flow below the instantaneous inspiratory flow during inspiration so that the oxygen concentration may be measured with the sensor described here.

Given that, in general, the inspiratory volume and expiratory volume are the same, it is possible measure the flow on expiration only.

When flow occurs out of the nose into the ambient environment, the measured fraction in the nose can be expressed as:

$${F}_{m} ( t ) = \frac{{F}_{o} ( t ) {Q}_{o} ( t ) + {F}_{E} ( t ) {Q}_{E} ( t )}{{Q}_{o} ( t ) + {Q}_{E} ( t )} \qquad ( 40 )$$

where:

Q_{E} is the expiratory flow rate

$${Q}_{E} = {Q}_{O} ( t ) \frac{{F}_{O} ( t ) - {F}_{m} ( t )}{{F}_{m} ( t ) - {F}_{E} ( t )} \qquad ( 41 )$$

F_{E} (t) cannot be measured directly however if we oscillate the oxygen flow then F_{E}(t) can be found using equation (17)

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

which can then be substituted into equation (41) to find the instantaneous expired flow. The frequency of oscillation is optionally greater than the breathing frequency.

Interpolation of similar can then give a continuous Q_{E}(t) which can be integrated to find the tidal volume, i.e.

$${V}_{Tidal} ( t ) = \int {Q}_{E} ( t ) dt \qquad ( 42 )$$

Oscillating the oxygen during expiration has the advantage that it does not affect the FiO2 of the patient.

In addition to calculating the F_{E}(t) from measured/known parameters, it could also be deduced from measuring FiO2 during inspiration and assuming a difference between an inspired and expired oxygen fraction (e.g. expired O2 fraction is 5% less than inspired). In addition it could also have an assumed value.

To calculate Equation 41, F_{E} need to be determined. However, it can be difficult to determine the parameter of a gas component (be it O2 fraction, CO2 fraction or other gas parameter) of the exhaled gas flow 13 when an apparatus gas flow 11 is provided to the patient, because the leakage (“leak gas flow”) 12 from the apparatus gas flow 11 from the respiratory apparatus 10 adds to the exhaled gas flow 13 to create a total gas outflow (“composite gas outflow”) 15 from the patient that is measured by the e.g. sensor 14.

Therefore, the exhaled gas flow 13 is not actually measured by sensor 14, but rather a composite gas outflow 15 that includes the leak flow 12 combined with the patient's exhaled gas flow. The leak gas flow 12 can dilute (e.g. in the case of measuring CO2 fraction) or increase (e.g. in the case of measuring O2 fraction) or more generally “alter” the gas component of the exhaled gas flow 13 being measured by the sensor 14, giving misleading information about the parameter of the gas component being obtained. This issue is exacerbated at high flow rates, e.g. when providing high flow therapy. So, instead of measuring the exhaled gas flow 13, the sensor is actually measuring a gas component of the composite gas outflow 15 which comprises the exhaled gas flow 13 and possibly at least a portion of the apparatus gas flow 11 (that is, the leak gas flow 12). The exhaled gas flow 13 is not actually measured, but rather the composite gas outflow is measured, so the apparent reading of the exhaled gas flow is not accurate. Note, the composite gas outflow 15 can comprises other gases too—e.g. those present in ambient air.

This situation is described in more detail in PCT/IB2021/052062, U.S. application 62/989,081 (from which PCT/IB2021/052062 claims priority) which is incorporated herein by reference in its entirety.

The derivation below can be used to provide an apparatus and method to determine an actual exhaled gas flow 13 parameter F_{E} of a desired gas component by measuring a parameter of the gas component in a composite gas outflow 15 at or near (“proximate”) the patient, and taking into account the effect of the leak gas flow 12 in the measure of the parameter and adjusting the measure accordingly (or otherwise using the measure and other information) to determine the parameter of the desired gas component in the actual exhaled gas flow 13 from the patient. The apparatus gas flow can be varied with a signature to assist to determine the parameter. Note, the composite gas outflow 15 can comprise other gases too (in addition to CO2 and O2)—e.g. those present in ambient air. The present embodiments described work in the presence of such additional gases.

The respiratory apparatus can comprise a flow source that is able to provide apparatus gas flow to the patient. The apparatus provides a time-varying apparatus gas flow, such that a time-varying parameter of the apparatus gas flow varies over time. This provides a signature that can be used to assist determine a gas parameter of actual exhaled gas flow 13. As possible examples, the time-varying parameter of the apparatus gas flow might be flow rate, or a gas proportion (such as gas fraction (e.g. O2 fraction) and/or gas partial pressure (e.g. O2 partial pressure)).

F_{E} is derived in one of the two following ways, depending on whether a time-varying flow rate or a time-varying O2 fraction of the apparatus gas flow 11 is used.

It should be noted that the constant k should be substituted by (1−k) in the following derivations (sections 7.2.1 and 7.2.2) for measurements at the nose. If the mouth is closed and measurements are at the nose then k=0 and (1−k)=1.

Note, varying oxygen fraction or flow rate can occur just during expiration to reduce any interruption to patient respiratory support.

### 7.2.1 Determining Respiratory Parameters Using Time-Varying Flow Rate of Apparatus Gas Flow

F_{E} can be determined as follows when using an apparatus gas flow 13 with a time-varying flow rate (for CO2 exhaled fraction—O2 exhaled fraction can use another equation as described later).

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

This is derived as follows:

Assuming that all or most of the gas expired by the patient exits the mouth, the volume fraction (Fₘ) of a gas measured at the mouth of a patient being provided with nasal high flow as a function of time may be expressed as:

$${F}_{m} ( t ) = \frac{{F}_{E} ( t ) {Q}_{E} ( t ) + k ( t ) {F}_{o} ( t ) {Q}_{o} ( t )}{{Q}_{E} ( t ) + k ( t ) {Q}_{o} ( t )} \qquad ( 1 )$$

where:

Qₒ is the flow rate of the apparatus gas flow 11

F_{O} is the volume fraction of the gas (e.g. O2) component in the apparatus gas flow 11 coming from the respiratory apparatus

Fₘ is the volume fraction of the gas (e.g. O2, CO2, N2, or other tracer gas) component in the composite gas outflow 15 from the patient.

F_{E} is the volume fraction of the gas (CO2 and/or O2,N2 or other tracer gas) in the exhaled patient gas flow 13 (volume fraction of expired gas)

k is the proportion of the apparatus gas flow 11 that comes out through the patient's mouth (and (1−k) is the proportion that goes through (e.g. goes out/exits) the nose)

Q_{E} is the flow rate of the patient exhaled gas flow

$$\begin{matrix}may be rearranged to find the ratio of the unknown quantities , k and {Q}_{E} : & \\ {F}_{m} ( t ) \cdot ( {Q}_{E} ( t ) + k ( t ) {Q}_{o} ( t ) ) = {F}_{E} ( t ) {Q}_{E} ( t ) + k ( t ) {F}_{o} ( t ) {Q}_{o} ( t ) & Equation ( 1 )\end{matrix}$$

$${Q}_{E} ( t ) \cdot ( {F}_{m} ( t ) - {F}_{E} ( t ) ) = k ( t ) ( {F}_{o} ( t ) {Q}_{o} ( t ) - {Q}_{o} ( t ) {F}_{m} ( t ) )$$

$$\frac{{Q}_{E} ( t )}{k ( t )} = \frac{{F}_{o} ( t ) {Q}_{o} ( t ) - {Q}_{o} ( t ) {F}_{m} ( t )}{{F}_{m} ( t ) - {F}_{E} ( t )} \qquad ( 2 )$$

$$\frac{{Q}_{E} ( t )}{k ( t )} = {Q}_{o} ( t ) \frac{{F}_{o} ( t ) - {F}_{m} ( t )}{{F}_{m} ( t ) - {F}_{E} ( t )} \qquad ( 3 )$$

For two samples taken at times t and t+Δt where Δt, the time between samples, is sufficiently short such that we can assume that during the patient's expiratory phase, the fraction of the (expired) gas component (volume fraction of the gas component measured in the patient composite gas outflow 15′ Fₘ), the patient exhaled gas flow flow rate (Q_{E}) and the proportion of the apparatus gas flow exiting the mouth (k) to be approximately constant then we can approximate:

$$\frac{{Q}_{E} ( t + Δ t )}{k ( t + Δ t )} ∼ \frac{{Q}_{E} ( t )}{k ( t )}$$

$${F}_{E} ( t + Δ t ) ∼ {F}_{E} ( t )$$

$$Then from ( 3 ) :$$

$${Q}_{o} ( t + Δ t ) \frac{{F}_{o} ( t + Δ t ) - {F}_{m} ( t + Δ t )}{{F}_{m} ( t + Δ t ) - {F}_{E} ( t )} \approx {Q}_{o} ( t ) \frac{{F}_{o} ( t ) - {F}_{m} ( t )}{{F}_{m} ( t ) - {F}_{E} ( t )}$$

$$Solving for {F}_{E :}$$

$$\frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \approx \frac{{F}_{m} ( t + Δ t ) - {F}_{E} ( t )}{{F}_{m} ( t ) - {F}_{E} ( t )}$$

$$( {F}_{m} ( t ) - {F}_{E} ( t ) ) \cdot \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \approx {F}_{m} ( t + Δ t ) - {F}_{E} ( t )$$

$${F}_{E} ( t ) \cdot \{ 1 - \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \} \approx {F}_{m} ( t + Δ t ) - {F}_{m} ( t ) \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}$$

$${F}_{E} ( t ) \approx \frac{{F}_{m} ( t + Δ t ) - {F}_{m} ( t ) \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}}{1 - \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}}$$

$${F}_{E} ( t ) \approx \frac{\begin{matrix}{Q}_{o} ( t ) {F}_{m} ( t + Δ t ) ( {F}_{o} ( t ) - {F}_{m} ( t ) ) - \\ {Q}_{o} ( t + Δ t ) {F}_{m} ( t ) ( {F}_{o} ( t + Δ t ) - {F}_{m} ( t + Δ t ) )\end{matrix}}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

This expression may be used to determine an exhaled gas flow 13 parameter such as the expired fraction of oxygen, carbon dioxide, nitrogen, helium and/or an anaesthetic agent such as sevoflurane.

In some configurations, a correction or compensation may be applied to equation (4) to obtain a better estimate of the parameter (F_{E}) of the gas flow component in the exhaled gas flow 13′. For example a correction or compensation may be applied to equation (4) to account for the assumption that the composite gas outflow and the proportion of the apparatus gas flow exiting the mouth to be approximately constant being not true. Such a correction or compensation may for example take into account a ratio of the mouth flow to patient interface flow as a function of patient interface flow rate, as described in the applicant's publication WO2017187391 or US2019/0150831, which are incorporated herein by reference in their entirety.

For measurements of CO₂, Fₒ(t)=Fₒ(t+Δt)˜0 and so equation (4) reduces to:

$${F}_{E} ( t ) ∼ \frac{{Q}_{o} ( t + Δ t ) {F}_{m} ( t ) {F}_{m} ( t + Δ t ) ) - {Q}_{o} ( t ) {F}_{m} ( t + Δ t ) {F}_{m} ( t )}{{Q}_{o} ( t + Δ t ) {F}_{m} ( t + Δ t ) - {Q}_{o} ( t ) {F}_{m} ( t )}$$

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 5 / 16 )$$

Above are expressions for oxygen and carbon dioxide in terms of known/measured quantities, Fₘ(t), Qₒ(t), Fₘ(t+Δt), Qₒ(t+Δt). The found F_{E}CO₂ can recover the carbon dioxide waveform.

### 7.2.2 Determining Respiratory Parameters Using Time-Varying Gas Fraction of Apparatus Gas Flow

Can be determined as follows when using an apparatus gas flow 11 with a time-varying gas fraction.

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

this is derived from equation 4, which is derived above, as follows:

If the oxygen fraction of the apparatus gas flow 11 is time-varied, but the flow rate of the apparatus gas flow 11 is constant, then

$${Q}_{O} ( t + Δ t ) = {Q}_{O} ( t )$$

and equation (4) reduces to:

$${F}_{E} ( t ) = \frac{{F}_{m} ( t + Δ t ) ( {F}_{o} ( t ) - {F}_{m} ( t ) ) - {F}_{m} ( t ) ( {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) ) )} \qquad ( 17 a )$$

Simplifying the top and bottom lines yields:

$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

7.3 Determining Expiratory Flow Rate and/or State Parameters During the Expiratory Cycle when the Patient's Mouth is Opened

This embodiment utilises equation 30 below to obtain Q_{E}—the expiratory flow rate (that is, flow rate of the exhaled gas-flow 13)

$${Q}_{E} ( t ) = k ( t ) {Q}_{o} ( t ) \frac{{F}_{o} ( t ) - {F}_{m} ( t )}{{F}_{m} ( t ) - {F}_{E} ( t )} \qquad ( 30 )$$

where

Q_{E} is the expiratory flow rate (exhaled gas-flow flow rate)

and the state parameters are

k is a value between 0 and 1 and is the proportion of the apparatus gas flow 11 that comes out through the mouth (and (1−k) is the fraction that goes through the nose).

It is assumed that substantially all patient expiration exits through the mouth when the mouth is open

Qₒ is the flow rate of the apparatus gas flow 11.

F_{O} is the volume fraction of the gas (e.g. O2, N2 or tracer gas) component in the apparatus gas flow 11 coming from the respiratory apparatus.

Fₘ is the volume fraction of the gas (e.g. O2, CO2, N2 or tracer gas) component in the composite gas outflow 15 from the patient.

F_{E} is volume fraction of the gas (e.g. CO2, O2, N2 or tracer gas) component in the exhaled patient gas flow 13 (volume fraction of expired gas).

Equation 30 is similar to equation 41 except that equation 30 has a k(t) term, whereas equation 41 has a (1−k(t)) term. This is because in equation 41, the mouth is closed, so the sensor 14 senses at the nose. In equation 30, the mouth is open and so the sensor 14 senses at the mouth.

This k(t) can be found as follows:

For example, using an interface sealed at one nostril and measuring flow out of the other nostril Qₘ:

$${Q}_{m} = ( 1 - k ) {Q}_{O} \qquad ( 28 )$$

This allows the value of k to be determined using:

$$k = 1 - \frac{{Q}_{m}}{{Q}_{O}} \qquad ( 29 )$$

Upon determination of the amount of high flow present at the mouth, we can find tidal volume.

Consider equation (14):

$$\frac{{Q}_{E} ( t )}{k ( t )} = {Q}_{o} ( t ) \frac{{F}_{o} ( t ) - {F}_{m} ( t )}{{F}_{m} ( t ) - {F}_{E} ( t )} \qquad ( 14 )$$

Then the expiratory flow can be expressed as:

$${Q}_{E} ( t ) = k ( t ) {Q}_{o} ( t ) \frac{{F}_{o} ( t ) - {F}_{m} ( t )}{{F}_{m} ( t ) - {F}_{E} ( t )} \qquad ( 30 )$$

k is the fraction of the apparatus gas flow that comes out through the mouth (and (1-k) is the fraction that goes through the nose)

Using equation 16 or 17 as previously derived, the volume fraction of expired gas, F_{E} can be determined, by varying the oxygen fraction or flow rate of the apparatus gas flow.

1. A method of determining a respiratory parameter of a patient during inspiration when receiving respiratory support and their mouth is closed comprising:
providing an apparatus gas flow with a flow rate and a gas proportion to a patient,
measuring a gas proportion of a composite gas inflow to the patient, determining a composite gas inflow flow rate using one or more of:
apparatus gas flow gas proportion,
apparatus gas flow flow rate,
composite gas inflow gas proportion,
ambient gas proportion,
and
from the composite gas inflow flow rate, determining one or more respiratory parameters.

2. A method according to claim 1 wherein respiratory parameters are one or more of:
tidal volume,
minute ventilation,
respiratory rate,
apnoea,
airway patency, and/or
peak flow rate.

3. A method according to claim 1 or 2 wherein the apparatus gas flow flow rate is less than inspiratory demand of the patient for at least part of inspiration.

4. A method according to any preceding claim wherein:
for some of inspiration, the apparatus gas flow is at a first flow rate that is at or above inspiratory demand, and
for some of inspiration, the apparatus gas flow is at a second or subsequent flow rate, each of the second or subsequent flow rate being below inspiratory demand.

5. A method according to claim 4, wherein the inspiratory demand is the inspiratory demand of the patient the method is performed on.

6. A method according any preceding claim wherein the apparatus gas flow flow rate is time-varying so that for some of inspiration the flow rate is at or above inspiratory demand and for some of inspiration the flow rate is below inspiratory demand.

7. A method according to claim 6 wherein the time-varying flow rate oscillates, and optionally oscillates at a frequency greater than breathing frequency.

8. A method according to any preceding claim wherein the gas proportion is gas fraction and/or gas partial pressure.

9. A method according to any preceding claim wherein the inspiratory demand is peak inspiratory demand.

10. A method according to any preceding claim wherein the composite gas inflow comprises the apparatus gas flow and ambient (entrained) gas flow.

11. A method according to any preceding claim wherein the gas is one or more of O2, CO2, N2 or a tracer gas.

12. A method according to any preceding claim wherein the composite gas inflow gas proportion is measured with a sensor at the nose.

13. A method according to any preceding claim wherein the method comprises determining the composite gas inflow flow rate using all of: apparatus gas flow gas proportion, apparatus gas flow flow rate, composite gas inflow gas proportion, ambient gas proportion.

14. A method according to any preceding claim wherein the composite gas inflow flow rate Q_{TOT} of the patient is found using
$${Q}_{Tot} ( t ) = {Q}_{o} ( t ) [ 1 + \frac{Fo ( t ) - {FiO}_{2} ( t )}{{FiO}_{2} ( t ) - {F}_{entrained} ( t )} ] \qquad ( 37 )$$
where
Q_{TOT} the flow rate of the (total inhaled) patient gas inflow (composite gas inflow) 17 (apparatus gas flow 11 and entrained air gas flow 16) into the patient, and as defined by:
$${Q}_{Tot} ( t ) = {Q}_{entrained} ( t ) + {Q}_{o} ( t ) \qquad ( 35 )$$
and the state parameters are:
Qₒ is the flow rate of the apparatus gas flow
F_{O} is the volume fraction of the gas (e.g. O2, N2 or tracer gas) component in the apparatus gas flow coming from the respiratory apparatus
Fₘ is the volume fraction of the gas (e.g. O2, N2 or tracer gas) component in the composite gas inflow (patient inspiratory gas flow) to the patient
F_{entrained} is the volume fraction of the gas (e.g. O2, N2 or tracer gas) component in the entrained air gas flow.

15. A method according to any preceding claim wherein the tidal volume can be defined as follows
$${V}_{Tidal} ( t ) = \int {Q}_{Tot} ( t ) dt \qquad ( 38 )$$

16. A method of determining a respiratory parameter of a patient during expiration when receiving respiratory support and their mouth is closed comprising:
providing an apparatus gas flow with a flow rate and a gas proportion, to a patient,
measuring a parameter of a gas present in a composite gas outflow from the patient,
determining an exhaled gas flow rate using one or more of:
apparatus gas flow gas proportion,
apparatus gas flow flow rate,
composite gas outflow gas parameter,
exhaled gas flow parameter, wherein the exhaled gas flow parameter is determined using the measured parameter of the gas present in the composite gas outflow and a time-varying flow rate or gas proportion,
and
from the exhaled gas flow rate, determining one or more respiratory parameters.

17. A method according to claim 16 wherein respiratory parameters are one or more of:
tidal volume,
minute ventilation,
respiratory rate,
apnoea,
airway patency, and/or
peak flow rate.

18. A method according to claim 16 or 17 wherein the gas proportion is gas fraction and/or gas partial pressure.

19. A method according to any one of claims 16 to 18 wherein the composite gas outflow comprises a leak gas flow and the exhaled gas flow.

20. A method according to any one of claims 16 to 19 wherein the composite gas outflow parameter comprises a gas proportion measured with a sensor at the nose.

21. A method according to any one of claims 16 to 20 wherein the gas is one or more of O2, CO2, N2 or a tracer gas.

22. A method according to any one of claims 16 to 21 wherein the method comprises determining the exhaled gas flow flow rate using all of: apparatus gas flow gas proportion, apparatus gas flow flow rate, composite gas outflow gas proportion, exhaled gas flow parameter.

23. A method according to any one of claims 16 to 22 wherein one of the flow rate and gas proportion are time-varying.

24. A method according any one of claims 16 to 23 wherein the apparatus gas flow flow rate or gas proportion oscillates.

25. A method according to claim 24 wherein the flow rate or gas proportion oscillates at a frequency greater than breathing frequency.

26. A method according to any one of claims 16 to 25 wherein the exhaled gas flow parameter is an exhaled gas flow gas proportion.

27. A method according to any one of claims 16 to 26 wherein the exhaled gas flow flow rate Q_{E} of the patient is found using:
$${Q}_{E} = {Q}_{O} ( t ) \frac{{F}_{O} ( t ) - {F}_{m} ( t )}{{F}_{m} ( t ) - {F}_{E} ( t )} \qquad ( 41 )$$
where
Q_{E} is the expiratory flow rate (exhaled gas-flow flow rate) and the state parameters are
Qₒ is the flow rate of the apparatus gas flow
F_{O} is the volume fraction of the gas (e.g. O2, N2 or tracer gas) component in the apparatus gas flow 11 coming from the respiratory apparatus
Fₘ is the volume fraction of the gas (e.g. O2, CO2, N2 or tracer gas) component in the composite gas outflow 15 from the patient
F_{E} is volume fraction of the gas (e.g. CO2, O2, N2 or tracer gas) in the exhaled patient gas flow 13 (volume fraction of expired gas).

28. A method according to claim 27 wherein, if using varying flow rate for apparatus gas flow and O2 fraction exhaled then
$${F}_{E} ( t ) \approx \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

29. A method according to claim 27 wherein, if using varying flow rate for apparatus gas flow and CO2 fraction exhaled then
$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

30. A method according to claim 27 wherein, if using varying oxygen fraction for apparatus gas flow and O2 fraction exhaled
$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

31. A method according to any one of claims 16 to 30 wherein the tidal volume can be defined as follows
$${V}_{Tidal} ( t ) = \int {Q}_{E} ( t ) dt \qquad ( 42 )$$

32. A method of determining a respiratory parameter of a patient during expiration when receiving respiratory support comprising:
providing an apparatus gas flow with a flow rate and a gas proportion, to a patient,
measuring a parameter of the gas present in a composite gas outflow from the patient,
determining a proportion of apparatus gas flow through the mouth and/or nose,
determining an exhaled gas flow rate using one or more of:
apparatus gas flow gas proportion,
apparatus gas flow flow rate,
composite gas outflow gas parameter,
proportion of the apparatus gas flow,
exhaled gas flow parameter, wherein the exhaled gas flow parameter is determined using the measured parameter of the gas present in the composite gas outflow and a time-varying flow rate or gas proportion of the apparatus gas flow,
and
from the exhaled gas flow rate, determining one or more respiratory parameters.

33. A method according to claim 1 wherein respiratory parameters are one or more of:
tidal volume,
minute ventilation,
respiratory rate,
apnoea,
airway patency, and/or
peak flow rate.

34. A method according to claim 1 or 2 wherein the gas proportion is gas fraction and/or gas partial pressure.

35. A method according to any one of claims 32 to 34 wherein the composite gas outflow comprises a leak gas flow and the exhaled gas flow.

36. A method according to any one of claims 32 to 35 wherein the composite gas outflow parameter is a gas proportion measured with a sensor at the mouth.

37. A method according to any one of claims 32 to 36 wherein the gas is one or more of O2, CO2, N2 or a tracer gas.

38. A method according to any one of claims 32 to 37 wherein the method comprises determining the exhaled gas flow flow rate using all of: apparatus gas flow gas proportion, apparatus gas flow flow rate, composite gas outflow gas proportion, exhaled gas flow parameter.

39. A method according to any one of claims 32 to 38 wherein one of the flow rate and gas proportion are time-varying.

40. A method according any one of claims 32 to 39 wherein the apparatus gas flow flow rate or gas proportion oscillates.

41. A method according to claim 40 wherein the flow rate oscillates at a frequency greater than breathing frequency.

42. A method according to any one of claims 32 to 41 wherein the exhaled gas flow parameter is an exhaled gas flow gas proportion.

43. A method according to any one of claims 32 to 42 wherein determining a proportion of apparatus gas flow through the mouth and/or nose comprises determining a proportion of apparatus gas flow through the mouth.

44. A method according to any one of claims 32 to 43 wherein the proportion of apparatus gas flow through the mouth is a constant k with a value between 0 and 1.

45. A method according to any one of claims 32 to 44 wherein the exhaled gas flow flow rate Q_{E} of the patient is found using:
$${Q}_{E} ( t ) = k ( t ) {Q}_{o} ( t ) \frac{{F}_{o} ( t ) - {F}_{m} ( t )}{{F}_{m} ( t ) - {F}_{E} ( t )} \qquad ( 30 )$$
where
Q_{E} is the expiratory flow rate (patient exhaled gas-flow flow rate)
and the state parameters are
k is the proportion of the apparatus gas flow 11 that comes out through the mouth (and (1−k) is the fraction that goes through the nose). It is a value between 0 and 1, inclusive
F_{O} is the volume fraction of the gas (e.g. O2, N2 or tracer gas) component in the apparatus gas flow 11 coming from the respiratory apparatus
Fₘ is the volume fraction of the gas (e.g. O2, CO2, N2 or tracer gas) component in the composite gas outflow 15 from the patient
F_{E} is volume fraction of the gas (e.g. CO2, O2, N2 or tracer gas) in the exhaled patient gas flow 13 (volume fraction of expired gas).

46. A method according any one of claims 32 to 45 wherein, if using varying flow rate for apparatus gas flow and O2 fraction exhaled then
$${F}_{E} ( t ) \approx \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

47. A method according any one of claims 32 to 46 wherein if using varying flow rate for apparatus gas flow and CO2 fraction exhaled then
$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 16 )$$

48. A method according any one of claims 32 to 47 wherein, if using varying oxygen fraction for apparatus gas flow and O2 fraction exhaled
$${F}_{E} ( t ) = \frac{{F}_{m} ( t + Δ t ) {F}_{o} ( t ) - {F}_{m} ( t ) {F}_{o} ( t + Δ t )}{{F}_{o} ( t ) - {F}_{o} ( t + Δ t ) + {F}_{m} ( t + Δ t ) - {F}_{m} ( t )} \qquad ( 17 )$$

49. A method according to any one of claims 32 to 48 wherein the tidal volume can be defined as follows
$${V}_{Tidal} ( t ) = \int {Q}_{E} ( t ) dt \qquad ( 42 )$$

50. A method of determining a respiratory parameter of a patient during expiration when receiving respiratory support comprising:
providing an apparatus gas flow with a flow rate and a gas proportion, to a patient,
measuring a parameter of the gas present in a composite gas outflow from the patient,
determining an exhaled gas flow rate using one or more of:
apparatus gas flow gas proportion,
apparatus gas flow flow rate,
composite gas outflow gas parameter,
exhaled gas flow parameter, wherein the exhaled gas flow parameter is determined using the measured parameter of the gas present in the composite gas outflow and a time-varying flow rate or gas proportion of the apparatus gas flow,
and
from the exhaled gas flow rate, determining one or more respiratory parameters.

51. A method according to claim 49 further comprising determining a proportion of apparatus gas flow through the mouth and/or nose, and wherein the exhaled gas flow parameter is determined using the measured parameter of the gas present in the composite gas outflow, a time-varying flow rate or gas proportion, and the proportion of apparatus gas flow through the mouth and/or nose.

52. A method of determining a respiratory parameter of a patient when receiving respiratory support comprising:
determining in any order if a patient is inspiring or expiring and whether the mouth is open or closed,
if the mouth is closed,
during inspiration, determining a respiratory parameter according to any one of claims 1 to 15,
during expiration, determining a respiratory parameter according to any one of claims 16 to 31, 50 if the mouth is open,
during expiration determining a respiratory parameter according
to any one of claims 32 to 49, 50, 51

53. An apparatus for providing respiratory support and determining a respiratory parameter comprising:
a flow generator
one or more sensors or inputs for one or more sensors placed at a mouth and/or nose of a patient
a controller configured to carry out the method of any of claims 1 to 52.

54. An apparatus according to claim 53 further comprising a humidifier

55. An apparatus according to claim 53 or 54 further wherein the apparatus has or connects to a non-sealing interface.
